(19) United States
(12) Patent Application Publication
(10) Pub. No.: US 2026/0140113 A1
(43) Pub. Date: May 21, 2026

(54) PROTEIN BIOMARKERS FOR USE IN LUPUS NEPHRITIS

(71) Applicants: Exagen Inc., Vista, CA (US); The Johns Hopkins University, Baltimore, MD (US)

(72) Inventors: Andrea Fava, Timonium, MD (US); Michelle Petri, Timonium, MD (US); Sepehr Taghavi, Vista, CA (US); Andrew Concoff, Vista, CA (US); Jacob Vasquez, Vista, CA (US); Tyler O'Malley, Oceanside, CA (US)

(21) Appl. No.: 19/391,614

(22) Filed: Nov. 17, 2025

Related U.S. Application Data
(60) Provisional application No. 63/721,272, filed on Nov. 15, 2024.

(51) Int. Cl. G01N 33/564; G01N 33/573; G01N 33/68

(52) U.S. Cl. CPC G01N 33/564; G01N 33/573; G01N 33/6863; G01N 33/6872; G01N 33/6893; G01N 2800/24

(57) ABSTRACT

Methods of detecting proteins in biological samples from human patients having lupus nephritis and the use of proteins detected in biological samples to treat, diagnose, and monitor lupus nephritis and long-term kidney functional capacity in human patients.

### CROSS REFERENCE TO RELATED APPLICATIONS

This application claims the benefit of priority to U.S. Application No. 63/721,272 filed Nov. 15, 2024, the disclosure of which is incorporated by reference herein in its entirety and for all purposes.

### STATEMENT AS TO RIGHTS TO INVENTIONS MADE UNDER FEDERALLY SPONSORED RESEARCH AND DEVELOPMENT

This invention was made with government support under grant no. AR069572 awarded by the National Institutes of Health. The government has certain rights in the invention.

### BACKGROUND

Systemic lupus erythematosus (SLE) is an autoimmune disease characterized by the production of autoantibodies to various self-antigens and the activation and deposition of complement components in affected tissues. The kidneys are most often affected leading to lupus nephritis, which can eventually result in kidney failure and death. The pernicious nature of lupus nephritis often makes it difficult to diagnose, as early symptoms are not typically noticeable to human patients. Currently, the only definitive diagnosis involves an invasive kidney biopsy, but the invasiveness of the procedure is an obstacle to early diagnosis. There is a need in the art for less invasive means of detecting lupus nephritis earlier in the disease course to allow physicians to intervene before irreversible kidney damage occurs. The disclosure is directed to this, as well as other, important ends.

### BRIEF SUMMARY

Provided herein is a method of detecting proteins in a human patient having lupus nephritis comprising detecting (a) an elevated level of a protein, relative to a control, in a biological sample obtained from the patient, wherein the protein comprises CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, azurocidin (AZU1), thyroglobulin, DBH, visfatin, DR6, DLL1, IGFBP-6, MIP-1d, IR-1Ra, Siglec-5, LIMP-2, ANGPTL4, ESAM, MCP-1, TIM-1, TIMP-1, NOTCH-3, ANG-4, B7-2, BAMBI, BLMH, C1qTNF1, CD155, CD229, CD27, CD40L, calsyntenin-1, caspase 7, CXADR, Cf10, contactin-4, cripto-1, DCTN1, Dkk-3, ephrin-B3, FABP6, FGF-5, GFR alpha-2, glypican 2, hepassocin, IL-13 R2, IL-22, IL-23, LDL R, LRRC4, legumain, MBL, MCP-2, MIP-3b, MMP-1, melan-A, nectin-1, neuropilin-1, PCDH17, PTH, Pref-1, SLPI, Shh-N, stabilin-2, TFF3, TIM-1, thrombospondin-5, aFGF, or a combination of two or more thereof; and/or (b) a reduced level of a protein, relative to a control, in the biological sample obtained from the patient, wherein the protein comprises C5a, FOLR2, PRX1, P-cadherin, NCAM-1, IFN-gamma R1, HNMT, galectin-4, fetuin B, CRTAM, MFRP, SULTIB1, angiostatin, C-myc, FGF-20, GDF-11, or a combination of two or more thereof.

Provided herein is a method of treating lupus nephritis in a human patient in need thereof comprising: detecting (a) an elevated level of a protein, relative to a control, in a biological sample obtained from the patient, wherein the protein comprises CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, azurocidin, thyroglobulin, DBH, visfatin, DR6, DLL1, IGFBP-6, MIP-1d, IR-1Ra, Siglec-5, LIMP-2, ANGPTL4, ESAM, MCP-1, TIM-1, TIMP-1, NOTCH-3, ANG-4, B7-2, BAMBI, BLMH, ClqTNF1, CD155, CD229, CD27, CD40L, calsyntenin-1, caspase 7, CXADR, Cf10, contactin-4, cripto-1, DCTN1, Dkk-3, ephrin-B3, FABP6, FGF-5, GFR alpha-2, glypican 2, hepassocin, IL-13 R2, IL-22, IL-23, LDL R, LRRC4, legumain, MBL, MCP-2, MIP-3b, MMP-1, melan-A, nectin-1, neuropilin-1, PCDH17, PTH, Pref-1, SLPI, Shh-N, stabilin-2, TFF3, TIM-1, thrombospondin-5, aFGF, or a combination of two or more thereof, and/or (b) a reduced level of a protein, relative to a control, in the biological sample obtained from the patient, wherein the protein comprises C5a, FOLR2, PRX1, P-cadherin, NCAM-1, IFN-gamma R1, HNMT, galectin-4, fetuin B, CRTAM, MFRP, SULTiB1, angiostatin, C-myc, FGF-20, GDF-11, or a combination of two or more thereof; and (ii) administering to the human patient an effective amount of a lupus nephritis therapeutic agent.

These and other embodiments of the disclosure are described herein.

### BRIEF DESCRIPTION OF THE DRAWINGS

FIGS. 1A-1E show the performance characteristics of 12 urinary protein biomarkers and conventional biomarkers in predicting an NIH activity index greater than 2 at baseline. FIG. 1A: receiver operating characteristics (ROC) curves, 5-fold cross-validation. FIG. 1B: receiver operating characteristics (ROC) curves, test set. FIG. 1C: relative feature importance from 12 urine proteins (in order from top to bottom: CD163, cathepsin S, FOLR2, CEACAM-1, PRTN3, P-cadherin, C5a, PRX1, catalase, IP-10, IP-16, NCAM-1). FIGS. 1D-1E: probabilities of activity index greater than 2 from 12 urine proteins for test set by lupus nephritis class. In FIGS. 1D-1E, the x-axis represents the lupus nephritis classification.

FIGS. 2A-2E show NIH activity score trajectories. The trajectories are stratified by 12-month renal response rates in all lupus nephritis categories (FIG. 2A); from proliferative lupus and mixed (FIG. 2B); and from membranous lupus nephritis (FIG. 2C). FIG. 2D shows the class change from V0 to V1 in proliferative and mixed lupus nephritis. High indicates a NIH Activity Index greater than 2. Low indicates a NIH Activity Index less than or equal to 2. FIG. 2E shows the T Test for delta V1 and V0 in proliferative and mixed lupus nephritis groups for responders vs. non-responders.

FIG. 3 shows clinical characteristics of the study cohort from the patients in FIGS. 1-2.

FIG. 4 illustrates how probability scores from the 12-protein model (CD163, cathepsin S, FOLR2, CEACAM-1, PRTN3, P-cadherin, C5a, PRX1, catalase, IP-10, IP-16, NCAM-1) predict a 40% glomerular filtration rate (GFR) loss or end-stage kidney disease (ESKD) more accurately than the urine protein-to creatinine ratio (UPCR) both in the remission range and in the non-remission range.

FIGS. 5A-5B illustrate how probability scores from the 12-protein model (CD163, cathepsin S, FOLR2, CEACAM-1, PRTN3, P-cadherin, C5a, PRX1, catalase, IP-10, IP-16, NCAM-1) predict a 40% GFR loss or ESKD more accurately than UPCR both in the remission range and in the non-remission range.

FIGS. 6A-6B show that the concentrations of urinary proteins at 12 months that predict future GFR loss >40% or progression to ESKD adjusted for UPCR (FIG. 6A) or UPCR and eGFR (FIG. 6B). The horizontal axis indicates the hazard ratio (magnitude of effect) and the vertical axis indicate the negative log 10 of the p value. Time-to-event analysis (Cox proportional hazards model).

FIG. 7 shows that the concentrations of many urinary proteins at 12 months that predict future GFR loss >40% or progression to ESKD. The horizontal axis indicates the hazard ratio (magnitude of effect) and the vertical axis indicate the negative log 10 of the p value. Time-to-event analysis (Cox proportional hazards model).

FIGS. 8A-8B show the performance characteristics of IL-16 and CD163 urinary protein biomarkers in predicting histologically active lupus nephritis. FIG. 8A: Receiver operating characteristic (ROC) analysis demonstrated that the Random Forest (RF) model effectively discriminated between patients with elevated NIH Activity Index scores (>2) and those with low activity (≤2). The model achieved an area under the ROC curve (AUC) of 0.791, indicating good overall classification performance, with a sensitivity of 0.69 and a specificity of 0.75. FIG. 8B shows the relative importance of different features in a predictive model, measured by mean decrease Gini across iterative cross-validation (CV) resampling runs. The mean decrease Gini for a feature is the average amount of Gini impurity reduction contributed by that feature across all splits and all trees in the random forest. Mean decrease Gini serves as a global importance score that reflects how often and how effectively a feature is used to make informative splits throughout the ensemble. Each boxplot represents one feature, with the black dots indicating individual resampling results. CD163 has the highest mean decrease Gini (MeanDecreaseGini) value, indicating it is the most influential feature for model performance. CD163 consistently demonstrates modest increased contribution to model predictive power relative to IL-16 in a two-marker RF model.

FIGS. 9A-9B show the performance characteristics of IL-16 and CD163 urinary protein biomarkers in predicting histologically active lupus nephritis. FIG. 9A: Receiver operating characteristic (ROC) analysis demonstrated that the logistic regression (LR) model was able to discriminate between patients with elevated NIH Activity Index scores (>2) and those with low activity (≤2). The model achieved an area under the ROC curve (AUC) of 0.716, with a sensitivity of 0.67 and a specificity of 0.73. FIG. 9B: Logistic regression feature importance ranked by Z-value weight. The plot displays the top contributing biomarkers CD163 and IL-16 ordered by the magnitude of their logistic regression Z-statistics. Larger absolute Z-values indicate stronger and more statistically influential coefficients, reflecting each marker's contribution to distinguishing patients with elevated NIH Activity Index scores (>2) and those with low activity (≤2). CD163 shows the highest importance based on its Z-value weight, followed by IL-16.

FIGS. 10A-10B show the performance characteristics of azurocidin, thyroglobulin, catalase, tenascin C, and IL-16 urinary protein biomarkers in predicting histologically active lupus nephritis. FIG. 10A: Receiver operating characteristic (ROC) analysis demonstrated that the Random Forest (RF) model effectively discriminated between patients with elevated NIH Activity Index scores (>2) and those with low activity (≤2). The model achieved an area under the ROC curve (AUC) of 0.819, with a sensitivity of 0.82 and a specificity of 0.78, indicating strong overall classification performance. FIG. 10B shows the relative importance of different features in a predictive model, measured by mean decrease Gini across iterative cross-validation (CV) resampling runs. The mean decrease Gini for a feature is the average amount of Gini impurity reduction contributed by that feature across all splits and all trees in the random forest. Mean decrease Gini serves as a global importance score that reflects how often and how effectively a feature is used to make informative splits throughout the ensemble. Each boxplot represents one feature, with the black dots indicating individual resampling results. IL-16 has the highest mean decrease Gini (MeanDecreaseGini) value, indicating it is the most influential feature for model performance. Tenascin C and catalase show strong importance. The other features provide low, but relevant insights. Features related to inflammatory and oxidative stress markers (IL-16, tenascin C) dominate the model, while others contribute less consistently. This indicates prioritizing these top features for predictive accuracy or biomarker selection Proteins from left to right in FIG. 10B are azurocidin, thyroglobulin, catalase, tenascin C, and IL-16.

FIGS. 11A-11B show the performance characteristics of tenascin C, IL-16, catalase, thyroglobulin, and azurocidin urinary protein biomarkers in predicting histologically active lupus nephritis. FIG. 11A: Receiver operating characteristic (ROC) analysis demonstrated that the logistic regression (LR) model was able to discriminate between patients with elevated NIH Activity Index scores (>2) and those with low activity (≤2). Area under the curve was 0.722 with a sensitivity and specificity of 0.66 and 0.67, respectively. FIG. 11B: Logistic regression feature importance ranked by Z-value weight. The plot displays the top five biomarkers (tenascin C, IL-16, catalase, thyroglobulin, and azurocidin) ordered by the magnitude of their logistic regression Z-statistics. Larger absolute Z-values indicate stronger and more statistically influential coefficients, highlighting features that contribute most prominently to distinguishing patients with elevated NIH Activity Index scores (>2) and those with low activity (≤2). Tenascin C, followed by IL-16, Catalase, Thyroglobulin, and Azurocidin were the most significant contributors to the model predictions. Proteins from left to right in FIG. 11B are tenascin C, IL-16, catalase, thyroglobulin, and azurocidin.

FIGS. 12A-12B show the performance characteristics of thyroglobulin, S100A8, catalase, tenascin C, and IL-16 urinary protein biomarkers in predicting histologically active lupus nephritis. FIG. 12A: Receiver operating characteristic (ROC) analysis demonstrated that the Random Forest (RF) model effectively discriminated between patients with elevated NIH Activity Index scores (>2) and those with low activity (≤2). Area under the curve was 0.823 with a sensitivity and specificity of 0.88 and 0.74, respectively. FIG. 12B shows the relative importance of different features in a predictive model, measured by mean decrease Gini across iterative cross-validation (CV) resampling runs. The mean decrease Gini for a feature is the average amount of Gini impurity reduction contributed by that feature across all splits and all trees in the random forest. Mean decrease Gini serves as a global importance score that reflects how often and how effectively a feature is used to make informative splits throughout the ensemble. Each boxplot represents one feature, with the black dots indicating individual resampling results. IL-16 has the highest mean decrease Gini (MeanDecreaseGini) value, indicating it is the most influential feature for model performance. Tenascin C and catalase show strong importance. The other features provide low, but relevant insights. Features related to inflammatory and oxidative stress markers (IL-16, tenascin C) dominate the model, while others contribute less consistently. This indicates prioritizing these top features for predictive accuracy or biomarker selection. Proteins from left to right in FIG. 12B are thyroglobulin, S100A8, catalase, tenascin C, and IL-16.

FIGS. 13A-13B show the performance characteristics of tenascin C, S100A8, IL-16, thyroglobulin, and catalase urinary protein biomarkers in predicting histologically active lupus nephritis. FIG. 13A: Receiver operating characteristic (ROC) analysis demonstrated that the logistic regression (LR) model was able to discriminate between patients with elevated NIH Activity Index scores (>2) and those with low activity (≤2). Area under the curve was 0.741 with a sensitivity and specificity of 0.77 and 0.66, respectively. FIG. 13B: Logistic regression feature importance ranked by Z-value weight. The plot highlights the top contributors (tenascin C, S100A8, IL-16, thyroglobulin, and catalase) ordered by the magnitude of their logistic regression Z-statistics. Larger absolute Z-values indicate stronger and more statistically meaningful coefficients, identifying biomarkers that exert the greatest influence in distinguishing patients with elevated NIH Activity Index scores (>2) and those with low activity (≤2). Proteins from top to bottom in FIG. 13B are tenascin C, S100A8, IL-16, thyroglobulin, and catalase.

FIGS. 14A-14B show the performance characteristics of DBH, thyroglobulin, vistafin, PRTN3, catalase, tenascin C, and IL-16 urinary protein biomarkers in predicting histologically active lupus nephritis. FIG. 14A: Receiver operating characteristic (ROC) analysis demonstrated that the Random Forest (RF) model effectively discriminated between patients with elevated NIH Activity Index scores (>2) and those with low activity (≤2). Area under the curve was 0.838 with a sensitivity and specificity of 0.8 and 0.798, respectively. FIG. 14B shows the relative importance of different features in a predictive model, measured by mean decrease Gini across iterative cross-validation (CV) resampling runs. The mean decrease Gini for a feature is the average amount of Gini impurity reduction contributed by that feature across all splits and all trees in the random forest. Mean decrease Gini serves as a global importance score that reflects how often and how effectively a feature is used to make informative splits throughout the ensemble. Each boxplot represents one feature, with the black dots indicating individual resampling results. IL-16 has the highest mean decrease Gini (MeanDecreaseGini) value, indicating it is the most influential feature for model performance. Tenascin C and catalase show strong importance. The other features provide low, but relevant insights. Features related to inflammatory and oxidative stress markers (IL-16, tenascin C) dominate the model, while others contribute less consistently. This indicates prioritizing these top features for predictive accuracy or biomarker selection, Proteins from left to right in FIG. 14B are DBH, thyroglobulin, vistafin, PRTN3, catalase, tenascin C, and IL-16 (in order from left to right).

FIGS. 15A-15B show the performance characteristics of thyroglobulin, visfatin, tenascin C, IL-16, DBH, PRTN3, and catalase urinary protein biomarkers in predicting histologically active lupus nephritis. FIG. 15A: Receiver operating characteristic (ROC) analysis demonstrated that the logistic regression (LR) model was able to discriminate between patients with elevated NIH Activity Index scores (>2) and those with low activity (≤2). Area under the curve was 0.733 with a sensitivity and specificity of 0.66 and 0.74, respectively. FIG. 15B: Logistic regression feature importance ranked by Z-value weight. The biomarkers thyroglobulin, visfatin, tenascin C, IL-16, DBH, PRTN3, and catalase are shown in descending order of importance based on the magnitude of their logistic regression Z-statistics. Larger absolute Z-values indicate more influential and statistically meaningful coefficients, highlighting features that contribute most strongly to distinguishing patients with elevated NIH Activity Index scores (>2) and those with low activity (≤2). Proteins in FIG. 15B from top to bottom are thyroglobulin, visfatin, tenascin C, IL-16, DBH, PRTN3, and catalase.

FIGS. 16A-16B show the performance characteristics of azurocidin, thyroglobulin, DBH, catalase, tenascin C, and IL-16 urinary protein biomarkers in predicting histologically active lupus nephritis. FIG. 16A: Receiver operating characteristic (ROC) analysis demonstrated that the Random Forest (RF) model effectively discriminated between patients with elevated NIH Activity Index scores (>2) and those with low activity (≤2). Area under the curve was 0.824 with a sensitivity and specificity of 0.88 and 0.754, respectively. FIG. 16B shows the relative importance of different features in a predictive model, measured by mean decrease Gini (MeanDecreaseGini) value across iterative cross-validation (CV) resampling runs. The mean decrease Gini for a feature is the average amount of Gini impurity reduction contributed by that feature across all splits and all trees in the random forest. Mean decrease Gini serves as a global importance score that reflects how often and how effectively a feature is used to make informative splits throughout the ensemble. Each colored boxplot represents one feature, with the black dots indicating individual resampling results. IL-18 has the highest mean decrease Gini (MeanDecreaseGini) value, indicating it is the most influential feature for model performance. Tenascin C and catalase also show strong importance, with median values around 9-10. DBH has moderate importance, while thyroglobulin and azurocidin are the least influential, with median values near 5-6. The spread of the boxplots reflects variability across CV runs; IL-18 shows a wider range, indicating ts importance may fluctuate depending on the resampling. Features related to inflammatory and oxidative stress markers (IL-16, catalase, tenascin C) dominate the model, while others contribute less consistently. This indicates prioritizing these top features for predictive accuracy or biomarker selection. The proteins in FIG. 16B from left to right are azurocidin, thyroglobulin, DBH, catalase, tenascin C, and IL-16.

FIGS. 17A-17B show the performance characteristics of IL-16, tenascin C, thyroglobulin, catalase, DBH, and azurocidin urinary protein biomarkers in predicting histologically active lupus nephritis. FIG. 17A: Receiver operating characteristic (ROC) analysis demonstrated that the logistic regression (LR) model was able to discriminate between patients with elevated NIH Activity Index scores (>2) and those with low activity (≤2). Area under the curve was 0.715 with a sensitivity and specificity of 0.61 and 0.72, respectively. FIG. 17B: Logistic regression feature importance ranked by Z-value weight. The biomarkers IL-16, tenascin C, thyroglobulin, catalase, DBH, and azurocidin are shown in descending order of importance based on the magnitude of their logistic regression Z-statistics. Larger absolute Z-values indicate more influential and statistically meaningful coefficients, highlighting the features that most strongly contribute to distinguishing patients with elevated NIH Activity Index scores (>2) and those with low activity (≤2). The proteins in FIG. 17B from top to bottom are IL-16, tenascin C, thyroglobulin, catalase, DBH, and azurocidin.

FIG. 18 is a Kaplan-Meier survival curve by risk group for predicting long-term kidney functional capacity with the biomarkers tenascin C, CD163, DR6, DLL1, CD206 (MMR), IGFBP-6, and IL-6. The Median C index is 0.77.

FIG. 19 is a Kaplan-Meier survival curve by risk group for predicting long-term kidney functional capacity with the biomarkers tenascin C, MIP-1d (CCL15), IL-1Ra, MMR (CD206), and IGFBP-6. The Median C index is 0.79.

FIG. 20 is a Kaplan-Meier survival curve by risk group for predicting long-term kidney functional capacity with the biomarkers tenascin C, LIMP-2, Siglec-5, ANGPTL4, and ESAM. The Median C index is 0.73.

FIG. 21 is a Kaplan-Meier survival curve by risk group for predicting long-term kidney functional capacity with the biomarkers tenascin C, MCP-1, TIM-1, TIMP-1, DLL1, NOTCH-3, and CD163. The Median C index is 0.77.

FIG. 22 is a Kaplan-Meier survival curve by risk group for predicting long-term kidney functional capacity with the biomarkers PRTN3, tenascin C, CD163, and thyroglobulin. The Median C index is 0.75.

### DETAILED DESCRIPTION

Unless defined otherwise, technical and scientific terms used herein have the same meaning as commonly understood by a person of ordinary skill in the art. Any methods, devices and materials similar or equivalent to those described herein can be used in the practice of this disclosure. The following definitions are provided to facilitate understanding of certain terms used frequently herein and are not meant to limit the scope of the present disclosure.

“Lupus nephritis,” as is well-known in the art, is a type of kidney disease caused by system lupus erythematosus (SLE). Lupus nephritis can be divided into 6 classes. Class I lupus nephritis is defined as minimal mesangial glomerulonephritis. Class II lupus nephritis is defined as mesangial proliferative glomerulonephritis. Class III lupus nephritis is defined as focal glomerulonephritis (e.g., focal lupus nephritis). Class IV lupus nephritis is defined as diffuse glomerulonephritis (e.g., diffuse lupus nephritis). Class V lupus nephritis is defined as membranous glomerulonephritis (e.g., membranous lupus nephritis). Class VI lupus nephritis is defined as advanced sclerotic. Class III and IV lupus nephritis can further be classified based on active and/or chronic lesions.

“Mixed lupus nephritis” is a type of lupus nephritis that combines features of at least two classes of lupus nephritis including (i) Class III/IV lesion: a proliferative lesion, and (ii) diffusely distributed membranous lesion: a class V lesion.

A “lupus nephritis therapeutic agent” refers to a therapeutic agent (e.g., compound, biologic, pharmaceutical composition) that when administered to a subject will have the intended therapeutic effect, e.g., treating or reducing the progression of lupus nephritis, or its symptoms or the intended therapeutic effect, e.g., treatment or amelioration of lupus nephritis, or their symptoms including any objective or subjective parameter of treatment such as abatement; remission; diminishing of symptoms or making the lupus nephritis more tolerable to the patient; slowing in the rate of degeneration or decline. In embodiments, the lupus nephritis therapeutic agent is an immunosuppressive agent, a B-cell inhibitor, an interferon antagonist, a glucocorticoid, or a combination of two or more thereof.

The terms “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. In embodiments, the proteins described herein are human proteins.

The phrase “specifically (or selectively) binds” to an antibody or “specifically (or selectively) immunoreactive with,” when referring to a protein or peptide refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The terms “isolate” or “isolated” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or HPLC. A protein that is the predominant species present in a preparation is substantially purified.

The term “about” means a range of values including the specified value, which a person of ordinary skill in the art would consider reasonably similar to the specified value. In embodiments, “about” means within a standard deviation using measurements generally acceptable in the art. In embodiments, “about” means a range extending to +/−10% of the specified value.

The singular terms “a,” “an,” and “the” include the plural reference unless the context clearly indicates otherwise. For example, the term “a protein” refers to one protein or more than one protein.

“Treating” or “treatment” as used herein and as well-understood in the art includes any approach for obtaining beneficial clinical results for a patient. Beneficial clinical results includes, but is not limited to, alleviation or amelioration of one or more symptoms of a disease (i.e., lupus nephritis), diminishment of the extent of the disease, stabilizing (i.e., not worsening) the disease, delaying or slowing progression of the disease, amelioration or palliation of the disease, and remission, whether partial or total and whether detectable or undetectable. Treatment may relieve the disease's symptoms, fully or partially remove the disease's underlying cause, shorten a disease's duration, or do a combination of these things. Treatment methods include administering to a subject a therapeutically effective amount of a lupus nephritis therapeutic agent. The administering step may comprise a single administration or a series of administrations. The length of the treatment period depends on a variety of factors, such as the severity of the condition, the age of the patient, the concentration of lupus nephritis therapeutic agent, the activity of the compositions used in the treatment, or a combination thereof. It will also be appreciated that the effective dosage of lupus nephritis therapeutic agent used for the treatment may increase or decrease over the course of a particular treatment regime. Changes in dosage may result and become apparent by standard diagnostic assays known in the art. Treating does not include preventing.

“Patient” or “patient in need thereof” or “subject” or “subject in need thereof” refers to a mammal suffering having SLE and/or lupus nephritis that can be treated by administration of therapeutic agents as provided herein. In embodiments, the mammal is a human. In embodiments, a patient is a human having SLE. In embodiments, a patient is a human suspected of having SLE. In embodiments, a patient is a human having SLE and lupus nephritis. In embodiments, a patient is a human suspected of having lupus nephritis. In embodiments, a patient is a human having SLE who is at risk of developing lupus nephritis. In embodiments, a patient has an NIH Activity Index greater than 2. In embodiments, a patient is a human having SLE and an NIH Activity Index greater than 2. In embodiments, a patient is a human having SLE, lupus nephritis, and an NIH Activity Index greater than 2.

“SLE patient” or “system lupus erythematosus patient” refers to a patient have SLE. In embodiments, the patient is a human having SLE.

An “effective amount” is an amount sufficient for a lupus nephritis therapeutic agent to accomplish a stated purpose relative to the absence of the lupus nephritis therapeutic agent (e.g. achieve the effect for which it is administered, treat lupus nephritis, reduce enzyme activity, increase enzyme activity, or reduce a signaling pathway). An example of an “effective amount” is an amount sufficient to contribute to the treatment, prevention, or reduction of a symptom or symptoms of SLE, which could also be referred to as a “therapeutically effective amount.” A “reduction” of a symptom means decreasing of the severity or frequency of the symptom, or elimination of the symptom. The exact amounts will depend on the purpose of the treatment, and will be ascertainable by prescribing information on commercially available lupus nephritis therapeutic agents or techniques known in the art.

The term “administering” is used in accordance with its plain and ordinary meaning and includes oral, topical, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, nasal, or transdermal). Parenteral administration includes, e.g., intravenous, intramuscular, intradermal, subcutaneous, intraperitoneal, and the like). In embodiments, the administering does not include administration of any therapeutic agent other than the recited therapeutic agent.

The term “diagnosis” is used in accordance with its plain and ordinary meaning and refers to an identification of a disease (e.g., lupus nephritis), to an identification of the likelihood of the presence of a disease (e.g., lupus nephritis), the identification of a patient who may benefit from treatment with any particular lupus nephritis therapeutic agent, or outcome in a subject.

The term “biomarker” refers to an indicator, e.g., a predictive, prognostic, and/or a pharmacodynamic indicator, which can be detected in a biological sample. The biomarker may serve as an indicator of a patient who is at risk of developing lupus nephritis, a patient who has lupus nephritis, a patient who should be treated for lupus nephritis, a patient who should be monitored for lupus nephritis, the likelihood a patient will respond to a lupus nephritis therapeutic treatment, or a particular subtype of a disease or disorder, characterized by certain molecular, pathological, histological, and/or clinical features. In embodiments, a biomarker is a gene or a set of genes (i.e., a biomarker gene). In embodiments, the biomarker is a protein expressed by the biomarker gene. In embodiments, the biomarker is a protein concentration or a level of a protein. In embodiments, the biomarker is RNA expressed by the biomarker gene. Biomarkers include, but are not limited to, polynucleotides (e.g., DNA, and/or RNA), polynucleotide copy number alterations (e.g., DNA copy numbers), proteins, polypeptides, or polypeptide and polynucleotide modifications (e.g., posttranslational modifications).

A biomarker, such as a protein as referred to herein, includes any of the recombinant or naturally-occurring forms of the protein or variants or homologs thereof that maintain the activity of the protein (e.g. within at least 50%, 80%, 90%, 95%, 96%, 97%, 98%, 99% or 100% activity compared to the protein). In embodiments, the variants or homologs have at least 90%, 95%, 96%, 97%, 98%, 99% or 100% amino acid sequence identity across the whole sequence or a portion of the sequence (e.g., a 20, 50, 100, 150 or 200 continuous amino acid portion) compared to the naturally occurring protein. In embodiments, the protein is substantially identical to the protein identified by its UniProt reference number or a variant or homolog having substantial identity thereto.

An “increased level” or “elevated level” of a protein is an amount or concentration of the protein that is higher than the level (e.g., amount or concentration) of the protein in a control. The control may be any control known in the art, such as those described herein. In embodiments, an “increased level” or “elevated level” of the protein compared to the control (when the level of the protein is greater than the corresponding control) is, for example, an increase in the amount or concentration of the protein that is about 5%, 10%, 15%, 20%, 25%, 30%, 40%, 50%, 60%, 70%, 80%, 90%, 95%, or greater relative to the control. In embodiments, an “increased level” or “elevated level” of the protein is an amount or concentration of the protein that is statistically significantly higher than the amount or concentration of protein in the control.

A “decreased level” or “reduced level” of a protein is an amount or concentration of the protein that is lower than the level (e.g., amount or concentration) of the protein in a control. The control may be any control known in the art, such as those described herein. In embodiments, a “decreased level” of the protein compared to the control (when the level of the protein is greater than the corresponding control) is, for example, a decrease in the amount or concentration of the protein that is about 5%, 10%, 15%, 20%, 25%, 30%, 40%, 50%, 60%, 70%, 80%, 90%, 95%, or lower relative to the control. In embodiments, a “decreased level” of the protein is an amount or concentration of the protein that is statistically significantly lower than the amount or concentration of protein in the control.

“Comparable” in reference to the level of the protein refers to an amount or concentration of the protein that is not increased relative to the control and is not decreased relative to the control. The control may be any control known in the art, such as those described herein. In embodiments, “comparable” is +/−25% of the level of the protein in a control. In embodiments, “comparable” is +/−20% of the level of the protein in a control. In embodiments, “comparable” is +/−15% of the level of the protein in a control. In embodiments, “comparable” is +/−10% of the level of the protein in a control. In embodiments, “comparable” is +/−5% of the level of the protein in a control. In embodiments, a level of the protein that is “comparable” or “not increased” or “not decreased” is an amount or concentration of the protein that is not statistically significantly different than the amount or concentration of the control.

“Control” is used in accordance with its plain ordinary meaning and refers to an assay, comparison, or experiment in which the subjects or reagents of the experiment are treated as in a parallel experiment except for omission of a procedure, reagent, or variable of the experiment. In embodiments, the control is used as a standard of comparison in evaluating experimental effects. In embodiments, the control is a protein level (e.g., amount or concentration) against which another protein level (e.g. the level of a protein described herein) is compared (e.g., to make a diagnostic (e.g., predictive and/or prognostic) and/or therapeutic determination.

In embodiments, a control is a healthy patient or a healthy population of patients. In embodiments, a healthy patient is a patient that does not have SLE. In embodiments, a healthy patient is a patient that does not have SLE or lupus nephritis. In embodiments, a healthy patient is a patient that has SLE but does not have lupus nephritis. In embodiments, the control is an average value from population of healthy patients. In embodiments, the control is a housekeeping gene. In embodiments, the control is a patient with histologically inactive lupus nephritis. In embodiments, the control is a patient with nonproliferative lupus nephritis. In embodiments, a control is a pre-assigned value, e.g., a cut-off value which was previously determined to significantly separate a first group of patients (e.g., patients with SLE) from a second group of patients (e.g., healthy patients). In embodiments, the cut-off value is the median or mean (preferably median) protein level in the reference population. A control can also be obtained from the same individual, e.g., from an earlier-obtained sample, prior to disease, or prior to treatment. One of skill will recognize that controls can be designed for assessment of any number of parameters. In embodiments, a control is a negative control. In embodiments, such as some embodiments relating to detecting the level of protein or a subset of genes/proteins, a control comprises the average amount of proteins in a population of subjects (e.g., with SLE) or in a healthy or general population. In embodiments, the control comprises an average amount (e.g. amount of protein) in a population in which the number of subjects (n) is 5 or more, 50 or more, 100 or more, 1,000 or more, and the like. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant. Other controls can be used in the methods described herein in order to confirm the accuracy of results and to confirm the absence of impurities or contamination of reagents.

### Methods

Provided herein is a method of detecting proteins in a patient having lupus nephritis comprising detecting (a) an elevated level of a protein, relative to a control, in a biological sample obtained from the patient, wherein the protein comprises CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, or a combination of two or more thereof, and/or (b) a reduced level of a protein, relative to a control, in the biological sample obtained from the patient, wherein the protein comprises C5a, FOLR2, PRX1, P-cadherin, NCAM-1, IFN-gamma R1, HNMT, galectin-4, fetuin B, CRTAM, MFRP, SULT1B1, or a combination of two or more thereof. In embodiments, the method of detecting proteins in a patient having lupus nephritis further comprises detecting (c) an elevated level of a protein, relative to a control, in a biological sample obtained from the patient, wherein the protein comprises ANG-4, B7-2, BAMBI, BLMH, ClqTNF1, CD155, CD229, CD27, CD40L, calsyntenin-1, caspase 7, CXADR, Cf10, contactin-4, cripto-1, DCTN1, DLL1, DR6, Dkk-3, ephrin-B3, FABP6, FGF-5, GFR alpha-2, glypican 2, hepassocin, IL-13 R2, IL-22, IL-23, LDL R, LRRC4, legumain, MBL, MCP-2, MIP-3b, MMP-1, melan-A, nectin-1, neuropilin-1, PCDH17, PTH, Pref-1, SLPI, Shh-N, siglec-5, stabilin-2, TFF3, TIM-1, thrombospondin-5, aFGF, or a combination of two or more thereof; and/or (d) a reduced level of a protein, relative to a control, in the biological sample obtained from the patient, wherein the protein comprises angiostatin, C-myc, FGF-20, GDF-11, or a combination of two or more thereof. In embodiments, the method is of detecting proteins in a patient having histologically active lupus nephritis. In embodiments, the method further comprises administering to the patient an effective amount of a lupus nephritis therapeutic agent. The protein comprises or consist of any proteins described herein, whether they are individual proteins or any combination of two or more proteins. Descriptions of the individual proteins and exemplary combinations of proteins are described in detail herein. Descriptions of the lupus nephritis are described in detail herein. In embodiments, the patient has an NIH Activity Index greater than 2.

Provided herein is a method of detecting proteins in a patient having lupus nephritis comprising detecting an elevated level of a protein, relative to a control, in a biological sample obtained from the patient, wherein the protein comprises CD163, IL-16, azurocidin, tenascin C, IL-16, catalase, thyroglobulin, S100A8, PRTN3, DBH, visfatin, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, or a combination of two or more thereof. In embodiments, the method is of detecting proteins in a patient having histologically active lupus nephritis. In embodiments, the method further comprises administering to the patient an effective amount of a lupus nephritis therapeutic agent. The protein comprises or consist of any proteins described herein, whether they are individual proteins or any combination of two or more proteins. Descriptions of the individual proteins and exemplary combinations of proteins are described in detail herein. Descriptions of the lupus nephritis are described in detail herein. In embodiments, the patient has an NIH Activity Index greater than 2.

Provided herein is a method of treating lupus nephritis in a patient in need thereof comprising: detecting (a) an elevated level of a protein, relative to a control, in a biological sample obtained from the patient, wherein the protein comprises CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, or a combination of two or more thereof; and/or (b) a reduced level of a protein, relative to a control, in the biological sample obtained from the patient, wherein the protein comprises C5a, FOLR2, PRX1, P-cadherin, NCAM-1, IFN-gamma R1, HNMT, galectin-4, fetuin B, CRTAM, MFRP, SULT1B1, or a combination of two or more thereof, and (ii) administering to the patient an effective amount of a lupus nephritis therapeutic agent. In embodiments, the patient has histologically active lupus nephritis. The protein comprises or consist of any proteins described herein, whether they are individual proteins or any combination of two or more proteins. Descriptions of the individual proteins and exemplary combinations of the proteins are described in detail herein. Descriptions of the lupus nephritis are described in detail herein. In embodiments, the patient has an NIH Activity Index greater than 2.

Provided herein is a method of method of treating lupus nephritis in a human patient in need thereof comprising: (i) detecting an elevated level of a protein, relative to a control, in a biological sample obtained from the patient, wherein the protein comprises CD163, IL-16, azurocidin, tenascin C, IL-16, catalase, thyroglobulin, S100A8, PRTN3, DBH, visfatin, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, or a combination of two or more thereof, and (ii) administering to the human patient an effective amount of a lupus nephritis therapeutic agent. In embodiments, the patient has histologically active lupus nephritis. The protein comprises or consist of any proteins described herein, whether they are individual proteins or any combination of two or more proteins. Descriptions of the individual proteins and exemplary combinations of the proteins are described in detail herein. Descriptions of the lupus nephritis are described in detail herein. In embodiments, the patient has an NIH Activity Index greater than 2.

Provided herein is a method of treating lupus nephritis in a patient in need thereof comprising administering to the patient an effective amount of a lupus nephritis therapeutic agent; wherein a biological sample obtained from the patient has (a) an elevated level of a protein, relative to a control, in a biological sample obtained from the patient, wherein the protein comprises CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, or a combination of two or more thereof, and/or (b) a reduced level of a protein, relative to a control, in the biological sample obtained from the patient, wherein the protein comprises C5a, FOLR2, PRX1, P-cadherin, NCAM-1, IFN-gamma R1, HNMT, galectin-4, fetuin B, CRTAM, MFRP, SULTIB1, or a combination of two or more thereof. In embodiments, the patient has histologically active lupus nephritis. The protein comprises or consist of any proteins described herein, whether they are individual proteins or any combination of two or more proteins. Descriptions of the individual proteins and exemplary combinations of the proteins are described in detail herein. Descriptions of the lupus nephritis are described in detail herein. In embodiments, the patient has an NIH Activity Index greater than 2.

Provided herein is a method of treating lupus nephritis in a patient in need thereof comprising administering to the patient an effective amount of a lupus nephritis therapeutic agent; wherein a biological sample obtained from the patient has an elevated level of a protein, relative to a control, in a biological sample obtained from the patient, wherein the protein comprises CD163, IL-16, azurocidin, tenascin C, IL-16, catalase, thyroglobulin, S100A8, PRTN3, DBH, visfatin, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, or a combination of two or more thereof. In embodiments, the patient has histologically active lupus nephritis. The protein comprises or consist of any proteins described herein, whether they are individual proteins or any combination of two or more proteins. Descriptions of the individual proteins and exemplary combinations of the proteins are described in detail herein. Descriptions of the lupus nephritis are described in detail herein. In embodiments, the patient has an NIH Activity Index greater than 2.

Provided herein is a method of predicting the probability of histologically active lupus nephritis in a patient in need thereof comprising detecting (a) an elevated level of a protein, relative to a control, in a biological sample obtained from the patient, wherein the protein comprises CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, or a combination of two or more thereof, and/or (b) a reduced level of a protein, relative to a control, in the biological sample obtained from the patient, wherein the protein comprises C5a, FOLR2, PRX1, P-cadherin, NCAM-1, IFN-gamma R1, HNMT, galectin-4, fetuin B, CRTAM, MFRP, SULTIB1, or a combination of two or more thereof; thereby predicting that the patient likely has histologically active lupus nephritis. In embodiments, histologically active lupus nephritis is an NIH Activity Index greater than 2. In embodiments, the method further comprises administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein. Descriptions of the lupus nephritis are described in detail herein.

Provided herein is a method of predicting histologically active lupus nephritis in a human patient comprising: (i) detecting an elevated level of a protein, relative to a control, in a biological sample obtained from the patient, wherein the protein comprises CD163, IL-16, azurocidin, tenascin C, IL-16, catalase, thyroglobulin, S100A8, PRTN3, DBH, visfatin, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, or a combination of two or more thereof; and (ii) predicting histologically active lupus nephritis in the human patient. In embodiments, histologically active lupus nephritis is an NIH Activity Index greater than 2. In embodiments, the method further comprises administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein. Descriptions of the lupus nephritis are described in detail herein.

Provided herein is a method of predicting long-term kidney functional capacity in a patient with lupus nephritis in need thereof comprising detecting (a) an elevated level of a protein, relative to a control, in a biological sample obtained from the patient, wherein the protein comprises CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, ANG-4, B7-2, BAMBI, BLMH, ClqTNF1, CD155, CD229, CD27, CD40L, calsyntenin-1, caspase 7, CXADR, Cf10, contactin-4, cripto-1, DCTN1, DLL1, DR6, Dkk-3, ephrin-B3, FABP6, FGF-5, GFR alpha-2, glypican 2, hepassocin, IL-13 R2, IL-22, IL-23, LDL R, LRRC4, legumain, MBL, MCP-2, MIP-3b, MMP-1, melan-A, nectin-1, neuropilin-1, PCDH17, PTH, Pref-1, SLPI, Shh-N, siglec-5, stabilin-2, TFF3, TIM-1, thrombospondin-5, aFGF, or a combination of two or more thereof, and/or (b) a reduced level of a protein, relative to a control, in the biological sample obtained from the patient, wherein the protein comprises C5a, FOLR2, PRX1, P-cadherin, NCAM-1, IFN-gamma R1, HNMT, galectin-4, fetuin B, CRTAM, MFRP, SULTIB1, angiostatin, C-myc, FGF-20, GDF-11, GITR L, PCK1, RGM-C, thrombospondin-2, ZAP70, or a combination of two or more thereof; thereby predicting long-term kidney functional capacity in the patient with lupus nephritis. In embodiments, predicting long-term kidney functional capacity is predicting loss of glomerular filtration rate over time. In embodiments, the method further comprises administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein. Descriptions of the lupus nephritis are described in detail herein. In embodiments, the patient has an NIH Activity Index greater than 2.

Provided herein is a method of predicting long-term kidney functional capacity in a human patient with lupus nephritis comprising: (i) detecting an elevated level of a protein, relative to a control, in a biological sample obtained from the patient, wherein the protein comprises CD163, IL-16, azurocidin, tenascin C, IL-16, catalase, thyroglobulin, S100A8, PRTN3, DBH, visfatin, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, or a combination of two or more thereof; and (ii) predicting long-term kidney functional capacity in the human patient with lupus nephritis. In embodiments, predicting long-term kidney functional capacity is predicting loss of glomerular filtration rate over time. In embodiments, the method further comprises administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein. Descriptions of the lupus nephritis are described in detail herein. In embodiments, the patient has an NIH Activity Index greater than 2.

Provided herein is a method of diagnosing lupus nephritis in a patient comprising detecting (a) an elevated level of a protein, relative to a control, in a biological sample obtained from the patient, wherein the protein comprises CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, or a combination of two or more thereof, and/or (b) a reduced level of a protein, relative to a control, in the biological sample obtained from the patient, wherein the protein comprises C5a, FOLR2, PRX1, P-cadherin, NCAM-1, IFN-gamma R1, HNMT, galectin-4, fetuin B, CRTAM, MFRP, SULT1B1, or a combination of two or more thereof, and diagnosing the patient with lupus nephritis. In embodiments, the method further comprises administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein. Descriptions of the lupus nephritis are described in detail herein. In embodiments, the patient has an NIH Activity Index greater than 2.

Provided herein is a method of diagnosing lupus nephritis in a human patient comprising: (i) detecting an elevated level of a protein, relative to a control, in a biological sample obtained from the patient, wherein the protein comprises CD163, IL-16, azurocidin, tenascin C, IL-16, catalase, thyroglobulin, S100A8, PRTN3, DBH, visfatin, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, or a combination of two or more thereof, and (ii) diagnosing the human patient with lupus nephritis. In embodiments, the method further comprises administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein. Descriptions of the lupus nephritis are described in detail herein. In embodiments, the patient has an NIH Activity Index greater than 2.

Provided herein is a method of monitoring a patient who is at risk of developing lupus nephritis comprising: (i) detecting (a) an elevated level of a protein, relative to a control, in a biological sample obtained from the patient, wherein the protein comprises CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, or a combination of two or more thereof, and/or (b) a reduced level of a protein, relative to a control, in the biological sample obtained from the patient, wherein the protein comprises C5a, FOLR2, PRX1, P-cadherin, NCAM-1, IFN-gamma R1, HNMT, galectin-4, fetuin B, CRTAM, MFRP, SULT1B1, or a combination of two or more thereof, (ii) detecting a level of a protein in a biological sample obtained from the patient at a second time point, wherein the second time point is later than the first time point; (iii) comparing the level of a protein at the first time point to the level of a protein at the second time point, wherein: (a) the patient has lupus nephritis or is at risk for developing lupus nephritis when the level of a protein at the second time point in (a) is elevated and/or in (b) is reduced when compared to the level of a protein at the first time point; or (b) the patient does not have lupus nephritis when the level of a protein at the second time point in (a) and/or (b) is comparable to the level of a protein at the first time point. In embodiments, the level of a protein at the second time point is (a) elevated and/or (b) reduced when compared to the level of a protein at the first time point, such that the patient has lupus nephritis or is at risk for developing lupus nephritis. In embodiments, the level of a protein at the second time point is (a) elevated and/or (b) reduced when compared to the level of a protein at the first time point, such that the patient has lupus nephritis. In embodiments, the level of a protein at the second time point is comparable to the level of a protein at the first time point, such that the patient does not have lupus nephritis. In embodiments, the level of a protein at the first time point is (a) not elevated and/or (b) not reduced relative to a control. In embodiments, the level of a protein at the second time point is compared to a control. In embodiments, the method further comprises administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein. Descriptions of the lupus nephritis are described in detail herein. In embodiments, the patient has an NIH Activity Index greater than 2.

Provided herein is a method of monitoring a human patient who is at risk of developing lupus nephritis comprising: (i) detecting a level of a protein, relative to a control, in a biological sample obtained from the patient, wherein the protein comprises CD163, IL-16, azurocidin, tenascin C, IL-16, catalase, thyroglobulin, S100A8, PRTN3, DBH, visfatin, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, or a combination of two or more thereof; (ii) detecting a level of a protein in a biological sample obtained from the human patient at a second time point, wherein the second time point is later than the first time point; (iii) comparing the level of the protein at the first time point to the level of the protein at the second time point, wherein: (a) the patient has lupus nephritis or is at risk for developing lupus nephritis when the level of the protein at the second time point in (i) is elevated when compared to the level of the protein at the first time point; or (b) the patient does not have lupus nephritis when the level of the protein at the second time point in (i) is comparable to the level of the protein at the first time point. In embodiments, the level of a protein at the second time point is compared to a control. In embodiments, the method further comprises administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein. Descriptions of the lupus nephritis are described in detail herein. In embodiments, the patient has an NIH Activity Index greater than 2.

Provided herein is a method of monitoring efficacy of treatment for lupus nephritis in a patient in need thereof comprising: (i) detecting (a) an elevated level of a protein, relative to a control, in a biological sample obtained from the patient, wherein the protein comprises CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, or a combination of two or more thereof, and/or (b) a reduced level of a protein, relative to a control, in the biological sample obtained from the patient, wherein the protein comprises C5a, FOLR2, PRX1, P-cadherin, NCAM-1, IFN-gamma R1, HNMT, galectin-4, fetuin B, CRTAM, MFRP, SULTIB1, or a combination of two or more thereof; (ii) administering to the patient an effective amount of a lupus nephritis therapeutic agent; (iii) detecting a level of a protein in a biological sample obtained from the patient at a second time point, wherein the second time point is later than the first time point; and (iv) determining that: (a) the treatment is effective when the level of a protein at the second time point in (a) is reduced and/or in (b) is elevated when compared to the level of a protein at the first time point, or (b) the treatment is not effective when the level of a protein at the second time point is comparable to the level of a protein at the first time point and/or the level of a protein at the second time point in (a) is reduced and/or in (b) is elevated when compared to the level of a protein at the first time point. In embodiments, the level of a protein at the second time point is reduced and/or elevated compared to the level of a protein at the first time point, indicating that the treatment is effective. In embodiments, the level of a protein at the second time point is comparable to or (a) higher and/or (b) lower than the level of a protein at the first time point, indicating that the treatment is not effective. In embodiments, the level of a protein at the second time point is comparable to the level of a protein at the first time point, indicating that the treatment is not effective. In embodiments, the level of a protein at the second time point is (a) higher and/or (b) lower than the level of a protein at the first time point, indicating that the treatment is not effective. In embodiments, the level of a protein at the first time point is (a) not elevated relative and/or (b) not reduced relative to a control. In embodiments, the method further comprises comparing the level of a protein at the second time point to a control. In embodiments, the method further comprises administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein. Descriptions of the lupus nephritis are described in detail herein. In embodiments, the patient has an NIH Activity Index greater than 2.

Provided herein is a method of monitoring efficacy of treatment for lupus nephritis in a human patient in need thereof comprising: (i) detecting an elevated level of a protein, relative to a control, in a biological sample obtained from the patient, wherein the protein comprises CD163, IL-16, azurocidin, tenascin C, IL-16, catalase, thyroglobulin, S100A8, PRTN3, DBH, visfatin, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, or a combination of two or more thereof, (ii) administering to the human patient an effective amount of a lupus nephritis therapeutic agent; (iii) detecting a level of the protein in a biological sample obtained from the human patient at a second time point, wherein the second time point is later than the first time point; and (iv) determining that (a) the treatment is effective when the level of the protein at the second time point in (i) is reduced when compared to the level of the protein at the first time point, or the treatment is not effective when the level of the protein at the second time point is comparable to the level of the protein at the first time point is comparable to or elevated when compared to the level of the protein at the first time point. In embodiments, the method further comprises comparing the level of a protein at the second time point to a control. In embodiments, the method further comprises administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein. Descriptions of the lupus nephritis are described in detail herein. In embodiments, the patient has an NIH Activity Index greater than 2.

Provided herein is a method of treating lupus nephritis in a patient in need thereof comprising: (i) selecting a patient having a diagnosis of lupus nephritis based on (a) a lupus nephritis risk score or (b) a test result showing on an elevated level of a protein, relative to a control, in a biological sample obtained from the patient, wherein the protein comprises CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, or a combination of two or more thereof, and/or a reduced level of a protein, relative to a control, in the biological sample obtained from the patient, wherein the protein comprises C5a, FOLR2, PRX1, P-cadherin, NCAM-1, IFN-gamma R1, HNMT, galectin-4, fetuin B, CRTAM, MFRP, SULT1B1, or a combination of two or more thereof, and (ii) treating the patient from step (i) by administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein.

Provided herein is a method of treating lupus nephritis in a patient in need thereof comprising: (i) receiving or obtaining (a) a lupus nephritis risk score or (b) a test result showing an elevated level of a protein, relative to a control, in a biological sample obtained from the patient, wherein the protein comprises CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, or a combination of two or more thereof; and/or a reduced level of a protein, relative to a control, in the biological sample obtained from the patient, wherein the protein comprises C5a, FOLR2, PRX1, P-cadherin, NCAM-1, IFN-gamma R1, HNMT, galectin-4, fetuin B, CRTAM, MFRP, SULTiB1, or a combination of two or more thereof; and (ii) diagnosing a patient with lupus nephritis based on the lupus nephritis risk score or the test result of the protein levels, monitoring a patient who is at risk of developing lupus nephritis based on the lupus nephritis risk score or the test result of the protein levels, monitoring efficacy of treatment for lupus nephritis in a patient based on the lupus nephritis risk score or the test result of the protein levels; and (iii) treating the patient from step (ii) by administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein.

Provided herein is a method of treating lupus nephritis in a patient in need thereof comprising: (i) receiving or obtaining a lupus nephritis risk score or a test result on a protein level, wherein the lupus nephritis risk score or the test result of the protein level is produced by a non-transitory computer-readable storage medium having instructions stored thereon which, when executed by a processor, causes the processor to perform an operation comprising applying an algorithm to the results of a method which comprises detecting an elevated level of a protein, relative to a control, in a biological sample obtained from the patient, wherein the protein comprises CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, or a combination of two or more thereof; and/or a reduced level of a protein, relative to a control, in the biological sample obtained from the patient, wherein the protein comprises C5a, FOLR2, PRX1, P-cadherin, NCAM-1, IFN-gamma R1, HNMT, galectin-4, fetuin B, CRTAM, MFRP, SULTiB1, or a combination of two or more thereof; and (ii) diagnosing a patient with lupus nephritis based on the lupus nephritis risk score or the test result of the protein level, monitoring a patient who is at risk of developing lupus nephritis based on the lupus nephritis risk score or the test result of the protein level, monitoring efficacy of treatment for lupus nephritis in a patient based on the lupus nephritis risk score or the test result of the protein level; and (iii) treating the patient from step (ii) by administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein.

Provided herein is a method of treating lupus nephritis in a patient in need thereof comprising: (i) selecting a patient having a diagnosis of lupus nephritis based on (a) a lupus nephritis risk score or (b) a test result showing on an elevated level of a protein, relative to a control, in a biological sample obtained from the patient, wherein the protein comprises CD163, IL-16, azurocidin, tenascin C, IL-16, catalase, thyroglobulin, S100A8, PRTN3, DBH, visfatin, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, or a combination of two or more thereof; and (ii) treating the patient from step (i) by administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein.

Provided herein is a method of treating lupus nephritis in a patient in need thereof comprising: (i) receiving or obtaining (a) a lupus nephritis risk score or (b) a test result showing an elevated level of a protein, relative to a control, in a biological sample obtained from the patient, wherein the protein comprises CD163, IL-16, azurocidin, tenascin C, IL-16, catalase, thyroglobulin, S100A8, PRTN3, DBH, visfatin, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, or a combination of two or more thereof; and (ii) diagnosing a patient with lupus nephritis based on the lupus nephritis risk score or the test result of the protein levels, monitoring a patient who is at risk of developing lupus nephritis based on the lupus nephritis risk score or the test result of the protein levels, monitoring efficacy of treatment for lupus nephritis in a patient based on the lupus nephritis risk score or the test result of the protein levels; and (iii) treating the patient from step (ii) by administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein.

Provided herein is a method of treating lupus nephritis in a patient in need thereof comprising: (i) receiving or obtaining a lupus nephritis risk score or a test result on a protein level, wherein the lupus nephritis risk score or the test result of the protein level is produced by a non-transitory computer-readable storage medium having instructions stored thereon which, when executed by a processor, causes the processor to perform an operation comprising applying an algorithm to the results of a method which comprises detecting an elevated level of a protein, relative to a control, in a biological sample obtained from the patient, wherein the protein comprises CD163, IL-16, azurocidin, tenascin C, IL-16, catalase, thyroglobulin, S100A8, PRTN3, DBH, visfatin, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, or a combination of two or more thereof, and (ii) diagnosing a patient with lupus nephritis based on the lupus nephritis risk score or the test result of the protein level, monitoring a patient who is at risk of developing lupus nephritis based on the lupus nephritis risk score or the test result of the protein level, monitoring efficacy of treatment for lupus nephritis in a patient based on the lupus nephritis risk score or the test result of the protein level; and (iii) treating the patient from step (ii) by administering to the patient an effective amount of a lupus nephritis therapeutic agent. The protein comprises or consist of any proteins described herein, whether they are individual proteins or any combination of two or more proteins. Descriptions of the individual proteins and exemplary combinations of the proteins are described in detail herein.

In embodiments of the methods described herein, a medical provider instructs a patient to obtain laboratory tests. The laboratory analyzes a biological sample provided by the patient to produce test results, e.g., levels of protein and/or lupus nephritis risk scores. The medical provider receives the test results from the laboratory or obtains the test results from a patient so that the medical provider can use the test results to treat a patient with lupus nephritis, diagnose a patient with lupus nephritis, monitor a patient who is at risk of developing lupus nephritis, or monitor efficacy of treatment for lupus nephritis in a patient. Receiving and obtaining can be used interchangeably herein and can refer to physically receiving/obtaining paper documents or receiving/obtaining files via an electronic device (e.g., computer, phone). Medical provider refers to any person or entity that provides medical services to a patient. In embodiments, the medical provider is a medical doctor, a nurse, a nurse practitioner, a physician's assistant, a hospital, a doctor's office, and the like.

### Protein Biomarkers

In embodiments of any of the methods described herein, the protein biomarkers comprise any of the proteins described herein.

In embodiments of the methods described herein, the protein comprises CEACAM-1, CD163, IP-10, C5a, P-cadherin, IL-16, cathepsin S, PRX1, NCAM-1, catalase, FOLR2, PRTN3, or a combination of two or more thereof. In embodiments, the protein comprises CD163. In embodiments, the protein comprises CD163 and cathepsin S. In embodiments, the protein comprises CD163 and FOLR2. In embodiments, the protein comprises CD163 and CEACAM-1. In embodiments, the protein comprises CD163, cathepsin S, and FOLR2. In embodiments, the protein comprises CD163, cathepsin S, and CEACAM-1. In embodiments, the protein comprises CD163, cathepsin S, FOLR2, and CEACAM-1. In embodiments, the protein comprises CD163, cathepsin S, FOLR2, CEACAM-1, and PRTN3. In embodiments, the protein comprises CD163, cathepsin S, FOLR2, CEACAM-1, PRTN3, and p-cadherin. In embodiments, the protein comprises CD163, cathepsin S, FOLR2, CEACAM-1, PRTN3, p-cadherin, and C5a. In embodiments, the protein comprises CD163, cathepsin S, FOLR2, CEACAM-1, PRTN3, p-cadherin, C5a, and PRX1. In embodiments, the protein comprises CD163, cathepsin S, FOLR2, CEACAM-1, PRTN3, p-cadherin, C5a, and catalase. In embodiments, the protein comprises CD163, cathepsin S, FOLR2, CEACAM-1, PRTN3, p-cadherin, C5a, PRX1, and catalase. In embodiments, the protein comprises CD163, cathepsin S, FOLR2, CEACAM-1, PRTN3, p-cadherin, C5a, PRX1, catalase, and IP-10. In embodiments, the protein comprises CD163, cathepsin S, FOLR2, CEACAM-1, PRTN3, p-cadherin, C5a, PRX1, catalase, IP-10, and IL-16. In embodiments, the protein comprises CD163, cathepsin S, FOLR2, CEACAM-1, PRTN3, p-cadherin, C5a, PRX1, catalase, IP-10, IL-16, and NCAM-1. In embodiments, any of the protein combinations described herein further comprise cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, SULTIB1, or a combination of two or more thereof. In embodiments, any of the protein combinations described herein further comprise cyclophilin A. In embodiments, any of the protein combinations described herein further comprise FKBP51. In embodiments, any of the protein combinations described herein further comprise S100A13. In embodiments, any of the protein combinations described herein further comprise galectin-1. In embodiments, any of the protein combinations described herein further comprise MMR. In embodiments, any of the protein combinations described herein further comprise S100A8. In embodiments, any of the protein combinations described herein further comprise CES1. In embodiments, any of the protein combinations described herein further comprise nidogen-1. In embodiments, any of the protein combinations described herein further comprise IL-6. In embodiments, any of the protein combinations described herein further comprise CrkL. In embodiments, any of the protein combinations described herein further comprise midkine. In embodiments, any of the protein combinations described herein further comprise MIP-1b. In embodiments, any of the protein combinations described herein further tenascin C. In embodiments, any of the protein combinations described herein further C1q R1. In embodiments, any of the protein combinations described herein further comprise PAM. In embodiments, any of the protein combinations described herein further comprise PCSK9. In embodiments, any of the protein combinations described herein further comprise ADAM8. In embodiments, any of the protein combinations described herein further comprise CXCL16. In embodiments, any of the protein combinations described herein further comprise nestin. In embodiments, any of the protein combinations described herein further comprise galectin-8. In embodiments, any of the protein combinations described herein further comprise IFN-gamma R1. In embodiments, any of the protein combinations described herein further comprise HNMT. In embodiments, any of the protein combinations described herein further comprise galectin-4. In embodiments, any of the protein combinations described herein further comprise fetuin B. In embodiments, any of the protein combinations described herein further comprise CRTAM. In embodiments, any of the protein combinations described herein further comprise MFRP. In embodiments, any of the protein combinations described herein further comprise SULTIB1. In embodiments, any of the protein combinations described herein further comprise ANG-4, B7-2, BAMBI, BLMH, ClqTNF1, CD155, CD229, CD27, CD40L, calsyntenin-1, caspase 7, CXADR, Cf10, contactin-4, cripto-1, DCTN1, DLL1, DR6, Dkk-3, ephrin-B3, FABP6, FGF-5, GFR alpha-2, glypican 2, hepassocin, IL-13 R2, IL-22, IL-23, LDL R, LRRC4, legumain, MBL, MCP-2, MIP-3b, MMP-1, melan-A, nectin-1, neuropilin-1, PCDH17, PTH, Pref-1, SLPI, Shh-N, siglec-5, stabilin-2, TFF3, TIM-1, thrombospondin-5, aFGF, angiostatin, C-myc, FGF-20, GDF-11, GITR L, PCK1, RGM-C, thrombospondin-2, ZAP70, or a combination of two or more thereof.

In embodiments of the methods described herein, the protein comprises (a) CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, or a combination of two or more thereof, and (b) C5a, FOLR2, PRX1, P-cadherin, NCAM-1, IFN-gamma R1, HNMT, galectin-4, fetuin B, CRTAM, MFRP, SULT1B1, or a combination of two or more thereof. In embodiments, the protein comprises (a) CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, or a combination of two or more thereof, or (b) C5a, FOLR2, PRX1, P-cadherin, NCAM-1, IFN-gamma R1, HNMT, galectin-4, fetuin B, CRTAM, MFRP, SULT1B1, or a combination of two or more thereof.

In embodiments of the methods described herein, the protein comprises (a) CD163, PRTN3, catalase, CEACAM-1, IP-10, IL-16, cathepsin S, or a combination of two or more thereof; and (b) a reduced level of a protein, relative to a control, in the biological sample obtained from the patient, wherein the protein comprises C5a, FOLR2, PRX1, P-cadherin, NCAM-1, or a combination of two or more thereof. In embodiments, the protein comprises (a) CD163, PRTN3, catalase, CEACAM-1, IP-10, IL-16, cathepsin S, or a combination of two or more thereof, or (b) a reduced level of a protein, relative to a control, in the biological sample obtained from the patient, wherein the protein comprises C5a, FOLR2, PRX1, P-cadherin, NCAM-1, or a combination of two or more thereof.

In embodiments of the methods described herein, the protein comprises CEACAM-1, CD163, IP-10, C5a, P-cadherin, IL-16, cathepsin S, PRX1, NCAM-1, catalase, FOLR2, and PRTN3. In embodiments of the methods described herein, the protein comprises CEACAM-1, CD163, IP-10, P-cadherin, IL-16, cathepsin S, PRX1, NCAM-1, catalase, and PRTN3.

In embodiments of the methods described herein, the proteins consist of CEACAM-1, CD163, IP-10, C5a, P-cadherin, IL-16, cathepsin S, PRX1, NCAM-1, catalase, FOLR2, and PRTN3. In embodiments of the methods described herein, the proteins consist of CEACAM-1, CD163, IP-10, P-cadherin, IL-16, cathepsin S, PRX1, NCAM-1, catalase, and PRTN3.

In embodiments of the methods described herein, the protein comprises: (i) CEACAM-1, CD163, IP-10, C5a, P-cadherin, IL-16, cathepsin S, PRX1, NCAM-1, catalase, FOLR2, PRTN3, or a combination of two or more thereof, and (ii) cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, SULTIB1, or a combination of two or more thereof. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, SULT1B1, or a combination of two or more thereof.

In embodiments of the methods described herein, the protein comprises: (i) CEACAM-1, CD163, IP-10, C5a, P-cadherin, IL-16, cathepsin S, PRX1, NCAM-1, catalase, FOLR2, and PRTN3, and (ii) cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, SULTIB1, or a combination of two or more thereof. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) cyclophilin A. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) FKBP51. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) S100A13. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) galectin-1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) MMR. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) S100A8. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) CES1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) nidogen-1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) IL-6. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) CrkL. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) midkine. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) MIP-1b. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) tenascin C. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) C1q R1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) PAM. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) PCSK9. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) ADAM8. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) CXCL16. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) nestin. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) galectin-8. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) IFN-gamma R1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) HNMT. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) galectin-4. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) fetuin B. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) CRTAM. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) MFRP. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) SULT1B1.

In embodiments of the methods described herein, the protein comprises: (i) CEACAM-1, CD163, IP-10, C5a, P-cadherin, IL-16, cathepsin S, PRX1, NCAM-1, catalase, FOLR2, PRTN3, or a combination of two or more thereof, and (ii) cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1.

In embodiments of the methods described herein, the protein comprises: (i) CEACAM-1, CD163, IP-10, C5a, P-cadherin, IL-16, cathepsin S, PRX1, NCAM-1, catalase, FOLR2, and PRTN3, and (ii) at least two proteins selected from the group consisting of cyclophilin A, FKBP51, S100A13, galectin-1, MMR, S100A8, CES1, nidogen-1, IL-6, CrkL, midkine, MIP-1b, tenascin C, CIq R1, PAM, PCSK9, ADAM8, CXCL16, nestin, galectin-8, IFN-gamma R1, HNMT, galectin-4, fetuin B, CRTAM, MFRP, and SULT1B1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) at least three proteins selected from the group consisting of cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) at least four proteins selected from the group consisting of cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) at least five proteins selected from the group consisting of cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) at least six proteins selected from the group consisting of cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) at least seven proteins selected from the group consisting of cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) at least eight proteins selected from the group consisting of cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) at least nine proteins selected from the group consisting of cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1.

In embodiments of the methods described herein, the protein comprises: (i) CEACAM-1, CD163, IP-10, C5a, P-cadherin, IL-16, cathepsin S, PRX1, NCAM-1, catalase, FOLR2, and PRTN3, and (ii) two proteins selected from the group consisting of cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) three proteins selected from the group consisting of cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) four proteins selected from the group consisting of cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) five proteins selected from the group consisting of cyclophilin A, FKBP51, S100A13, galectin-1, MMR (CD206), S100A8, CES1, nidogen-1, IL-6, and CrkL. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) six proteins selected from the group consisting of cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) seven proteins selected from the group consisting of cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) eight proteins selected from the group consisting of cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1. In embodiments, the protein comprises: (i) CEACAM-1, CD163, IP-10, C5a, P-cadherin, IL-16, cathepsin S, PRX1, NCAM-1, catalase, FOLR2, and PRTN3, and (ii) nine proteins selected from the group consisting of cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1.

In embodiments of the methods described herein, the protein comprises CEACAM-1, CD163, IP-10, C5a, P-cadherin, IL-16, cathepsin S, PRX1, NCAM-1, catalase, FOLR2, PRTN3, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1.

In embodiments of the methods described herein, the proteins consist of CEACAM-1, CD163, IP-10, C5a, P-cadherin, IL-16, cathepsin S, PRX1, NCAM-1, catalase, FOLR2, PRTN3, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1.

In embodiments of the methods described herein, the protein comprises (i) CEACAM-1, CD163, IP-10, C5a, P-cadherin, IL-16, cathepsin S, PRX1, NCAM-1, catalase, FOLR2, PRTN3, or a combination of two or more thereof, and (ii) CXCL16, tenascin C, galectin-1, galectin-4, C1q R, ADAM8, CXCL16, S100A13, FKBP51, CES1, MMR, or a combination of two or more thereof. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) CXCL16, tenascin C, galectin-1, galectin-4, C1q R, ADAM8, CXCL16, S100A13, FKBP51, CES1, MMR, or a combination of two or more thereof.

In embodiments of the methods described herein, the protein comprises: (i) CEACAM-1, CD163, IP-10, C5a, P-cadherin, IL-16, cathepsin S, PRX1, NCAM-1, catalase, FOLR2, PRTN3, and (ii) at least two proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) at least three proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) at least four proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) at least five proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) at least six proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) at least seven proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) at least eight proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) at least nine proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) at least ten proteins selected from the group consisting of CXCL16, tenascin C, galectin-1, galectin-4, C1q R, ADAM8, CXCL16, S100A13, FKBP51, CES1, and MMR.

In embodiments of the methods described herein, the protein comprises: (i) CEACAM-1, CD163, IP-10, C5a, P-cadherin, IL-16, cathepsin S, PRX1, NCAM-1, catalase, FOLR2, PRTN3, and (ii) two proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) three proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) four proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) five proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) six proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) seven proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) eight proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) nine proteins selected from the group consisting of CXCL16, tenascin C, galectin-1, galectin-4, C1q R, ADAM8, CXCL16, S100A13, FKBP51, CES1, and MMR. In embodiments, the protein comprises: (i) CEACAM-1, CD163, IP-10, C5a, P-cadherin, IL-16, cathepsin S, PRX1, NCAM-1, catalase, FOLR2, PRTN3, and (ii) ten proteins selected from the group consisting of CXCL16, tenascin C, galectin-1, galectin-4, C1q R, ADAM8, CXCL16, S100A13, FKBP51, CES1, and MMR.

In embodiments, the protein comprises CEACAM-1, CD163, IP-10, C5a, P-cadherin, IL-16, cathepsin S, PRX1, NCAM-1, catalase, FOLR2, PRTN3, CXCL16, tenascin C, galectin-1, galectin-4, C1q R, ADAM8, CXCL16, S100A13, FKBP51, CES1, and MMR. In embodiments, the protein comprises CEACAM-1, CD163, IP-10, P-cadherin, IL-16, cathepsin S, PRX1, NCAM-1, catalase, PRTN3, CXCL16, tenascin C, galectin-1, galectin-4, C1q R, ADAM8, CXCL16, S100A13, FKBP51, CES1, and MMR.

In embodiments, the proteins consist of CEACAM-1, CD163, IP-10, C5a, P-cadherin, IL-16, cathepsin S, PRX1, NCAM-1, catalase, FOLR2, PRTN3, CXCL16, tenascin C, galectin-1, galectin-4, C1q R, ADAM8, CXCL16, S100A13, FKBP51, CES1, and MMR. In embodiments, the proteins consist of CEACAM-1, CD163, IP-10, P-cadherin, IL-16, cathepsin S, PRX1, NCAM-1, catalase, PRTN3, CXCL16, tenascin C, galectin-1, galectin-4, C1q R, ADAM8, CXCL16, S100A13, FKBP51, CES1, and MMR.

In embodiments of the methods described herein, the protein comprises cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, SULT1B1, or a combination of two or more thereof. In embodiments, the protein comprises cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1. In embodiments, the proteins consist of cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1.

In embodiments of the methods described herein, the protein comprises: (i) CEACAM-1, CD163, IP-10, C5a, P-cadherin, IL-16, cathepsin S, PRX1, NCAM-1, catalase, FOLR2, PRTN3, or a combination of two or more thereof, (ii) cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, SULT1B1, or a combination of two or more thereof (iii) ANG-4, B7-2, BAMBI, BLMH, ClqTNF1, CD155, CD229, CD27, CD40L, calsyntenin-1, caspase 7, CXADR, Cf10, contactin-4, cripto-1, DCTN1, DLL1, DR6, Dkk-3, ephrin-B3, FABP6, FGF-5, GFR alpha-2, glypican 2, hepassocin, IL-13 R2, IL-22, IL-23, LDL R, LRRC4, legumain, MBL, MCP-2, MIP-3b, MMP-1, melan-A, nectin-1, neuropilin-1, PCDH17, PTH, Pref-1, SLPI, Shh-N, siglec-5, stabilin-2, TFF3, TIM-1, thrombospondin-5, aFGF, angiostatin, C-myc, FGF-20, GDF-11, GITR L, PCK1, RGM-C, thrombospondin-2, ZAP70, or a combination of two or more thereof. In embodiments, the protein comprises: (i) CEACAM-1, CD163, IP-10, C5a, P-cadherin, IL-16, cathepsin S, PRX1, NCAM-1, catalase, FOLR2, and PRTN3, (ii) cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, SULT1B1, or a combination of two or more thereof, and (iii) ANG-4, B7-2, BAMBI, BLMH, ClqTNF1, CD155, CD229, CD27, CD40L, calsyntenin-1, caspase 7, CXADR, Cf10, contactin-4, cripto-1, DCTN1, DLL1, DR6, Dkk-3, ephrin-B3, FABP6, FGF-5, GFR alpha-2, glypican 2, hepassocin, IL-13 R2, IL-22, IL-23, LDL R, LRRC4, legumain, MBL, MCP-2, MIP-3b, MMP-1, melan-A, nectin-1, neuropilin-1, PCDH17, PTH, Pref-1, SLPI, Shh-N, siglec-5, stabilin-2, TFF3, TIM-1, thrombospondin-5, aFGF, angiostatin, C-myc, FGF-20, GDF-11, GITR L, PCK1, RGM-C, thrombospondin-2, ZAP70, or a combination of two or more thereof. In embodiments, the protein comprises (i) CEACAM-1, CD163, IP-10, C5a, P-cadherin, IL-16, cathepsin S, PRX1, NCAM-1, catalase, FOLR2, PRTN3, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, SULTIB1, and (ii) ANG-4, B7-2, BAMBI, BLMH, ClqTNF1, CD155, CD229, CD27, CD40L, calsyntenin-1, caspase 7, CXADR, Cf10, contactin-4, cripto-1, DCTN1, DLL1, DR6, Dkk-3, ephrin-B3, FABP6, FGF-5, GFR alpha-2, glypican 2, hepassocin, IL-13 R2, IL-22, IL-23, LDL R, LRRC4, legumain, MBL, MCP-2, MIP-3b, MMP-1, melan-A, nectin-1, neuropilin-1, PCDH17, PTH, Pref-1, SLPI, Shh-N, siglec-5, stabilin-2, TFF3, TIM-1, thrombospondin-5, aFGF, angiostatin, C-myc, FGF-20, GDF-11, GITR L, PCK1, RGM-C, thrombospondin-2, ZAP70, or a combination of two or more thereof.

In embodiments of the methods described herein, the protein comprises tenascin C. In embodiments, the protein comprises tenascin C and a protein selected from the group consisting of CD163, PRTN3, and MMR. In embodiments, the protein comprises tenascin C and CD163. In embodiments, the protein comprises tenascin C and PRTN3. In embodiments, the protein comprises tenascin C and MMR. In embodiments, the protein comprises tenascin C and two proteins selected from the group consisting of CD163, PRTN3, and MMR. In embodiments, the protein comprises tenascin C, CD163, and PRTN3. In embodiments, the protein comprises tenascin C, CD163, and MMR. In embodiments, the protein comprises tenascin C, PRTN3, and MMR. In embodiments, the protein comprises tenascin C, CD163, PRTN3, and MMR. In embodiments, the protein comprises tenascin C, CD163, PRTN3, MMR, or a combination of two or more thereof.

In embodiments of the methods described herein, the protein comprises CD163, IL-16, azurocidin, tenascin C, IL-16, catalase, thyroglobulin, S100A8, PRTN3, DBH, visfatin, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, or a combination of two or more thereof. In embodiments, the protein comprises IL-16, CD163, azurocidin (AZU1), thyroglobulin, catalase, tenascin C, S100A8, DBH, vistafin, PRTN3, or a combination of two or more thereof. In embodiments, the protein comprises tenascin C, CD163, DR6, DLL1, MMR, IGFBP-6, IL-6, MIP-1d, IL-1Ra, LIMP-2, Siglec-5, ANGPTL4, and ESAM, MCP-1, TIM-1, TIMP-1, NOTCH-3, PRTN3, thyroglobulin, or a combination of two or more thereof.

In embodiments of the methods of predicting histologically active lupus nephritis described herein, the protein comprises IL-16, CD163, azurocidin (AZU1), thyroglobulin, catalase, tenascin C, S100A8, DBH, vistafin, PRTN3, or a combination of two or more thereof.

In embodiments of the methods of predicting long-term kidney functional capacity described herein, the protein comprises tenascin C, CD163, DR6, DLL1, MMR, IGFBP-6, IL-6, MIP-1d, IL-1Ra, LIMP-2, Siglec-5, ANGPTL4, and ESAM, MCP-1, TIM-1, TIMP-1, NOTCH-3, PRTN3, thyroglobulin, or a combination of two or more thereof.

In embodiments of the methods described herein, the protein comprises IL-16, CD163, azurocidin, thyroglobulin, catalase, tenascin C, S100A8, DBH, vistafin, PRTN3, or a combination of two or more thereof. In embodiments, the protein comprises IL-16, CD163, azurocidin, thyroglobulin, catalase, tenascin C, S100A8, DBH, vistafin, and PRTN3. In embodiments, the protein comprises IL-16, CD163, catalase, tenascin C, S100A8, and PRTN3. In embodiments, the protein comprises CD163 and IL-16. In embodiments, the protein comprises azurocidin, tenascin C, IL-16, catalase, and thyroglobulin. In embodiments, the protein comprises S100A8, tenascin C, IL-16, catalase, and thyroglobulin. In embodiments, the protein comprises PRTN3, tenascin C, catalase, IL-16, thyroglobulin, DBH, and visfatin. In embodiments, the protein comprises azurocidin, tenascin C, IL-16, catalase, thyroglobulin, and DBH.

In embodiments of the methods described herein, the protein comprises tenascin C, CD163, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, PRTN3, thyroglobulin, or a combination of two or more thereof. In embodiments, the protein comprises tenascin C, CD163, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, PRTN3, and thyroglobulin. In embodiments, the protein comprises tenascin C, CD163, DR6, DLL1, MMR, IGFBP-6, and IL-6. In embodiments, the protein comprises tenascin C, MIP-1d, IL-1Ra, MMR, and IGFBP-6. In embodiments, the protein comprises tenascin C, LIMP-2, Siglec-5, ANGPTL4, and EASM. In embodiments, the protein comprises tenascin C, MCP-1, TIM-1, TIMP-1, DLL1, NOTCH-3, and CD163. In embodiments, the protein comprises PRTN3, tenascin C, CD163, and thyroglobulin.

In embodiments of the methods described herein, the proteins comprise at least one protein in Table 5. In embodiments, the proteins comprise at least 2 proteins in Table 5. In embodiments, the proteins comprise at least 3 proteins in Table 5. In embodiments, the proteins comprise at least 4 proteins in Table 5. In embodiments, the proteins comprise at least 5 proteins in Table 5. In embodiments, the proteins comprise at least 6 proteins in Table 5. In embodiments, the proteins comprise at least 7 proteins in Table 5. In embodiments, the proteins comprise at least 8 proteins in Table 5. In embodiments, the proteins comprise at least 9 proteins in Table 5. In embodiments, the proteins comprise at least 10 proteins in Table 5. In embodiments, the proteins comprise at least 11 proteins in Table 5. In embodiments, the proteins comprise at least 12 proteins in Table 5. In embodiments, the proteins comprise at least 13 proteins in Table 5. In embodiments, the proteins comprise at least 14 proteins in Table 5. In embodiments, the proteins comprise at least 15 proteins in Table 5. In embodiments, the proteins comprise at least 16 proteins in Table 5. In embodiments, the proteins comprise at least 17 proteins in Table 5. In embodiments, the proteins comprise at least 18 proteins in Table 5. In embodiments, the proteins comprise at least 19 proteins in Table 5. In embodiments, the proteins comprise at least 20 proteins in Table 5.

In embodiments of the methods described herein, the proteins comprise one protein in Table 5. In embodiments, the proteins comprise 2 protein in Table 5. In embodiments, the proteins comprise 3 proteins in Table 5. In embodiments, the proteins comprise 4 proteins in Table 5. In embodiments, the proteins comprise 5 proteins in Table 5. In embodiments, the proteins comprise 6 proteins in Table 5. In embodiments, the proteins comprise 7 proteins in Table 5. In embodiments, the proteins comprise 8 proteins in Table 5. In embodiments, the proteins comprise 9 proteins in Table 5. In embodiments, the proteins comprise 10 proteins in Table 5. In embodiments, the proteins comprise 11 proteins in Table 5. In embodiments, the proteins comprise 12 proteins in Table 5. In embodiments, the proteins comprise 13 proteins in Table 5. In embodiments, the proteins comprise 14 proteins in Table 5. In embodiments, the proteins comprise 15 proteins in Table 5. In embodiments, the proteins comprise 16 proteins in Table 5. In embodiments, the proteins comprise 17 proteins in Table 5. In embodiments, the proteins comprise 18 proteins in Table 5. In embodiments, the proteins comprise 19 proteins in Table 5. In embodiments, the proteins comprise 20 proteins in Table 5.

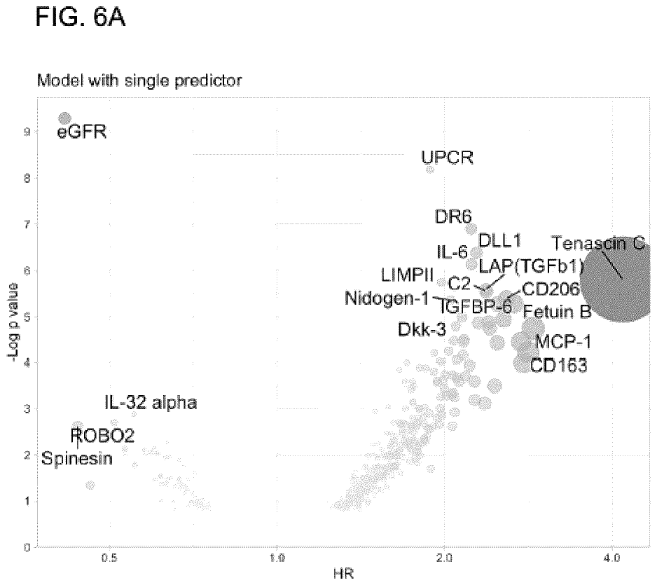

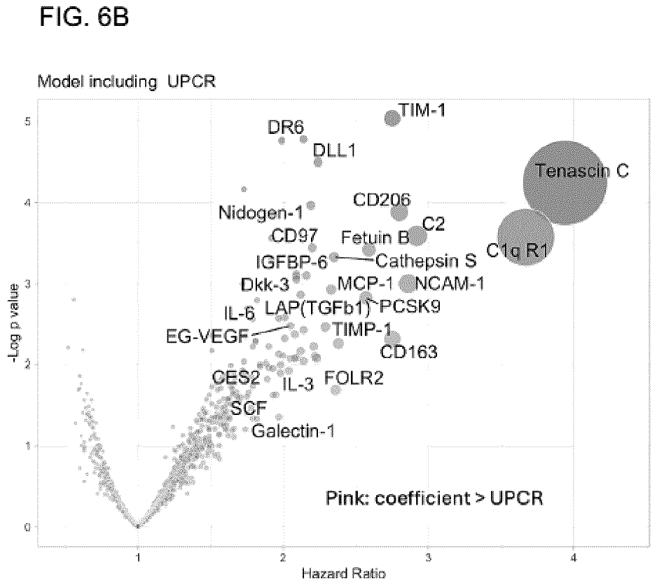

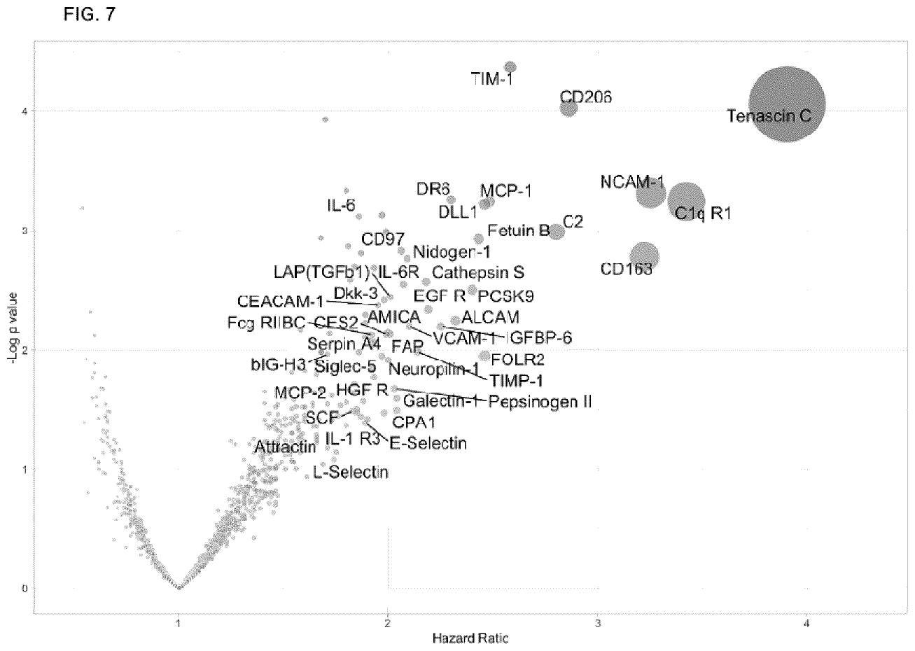

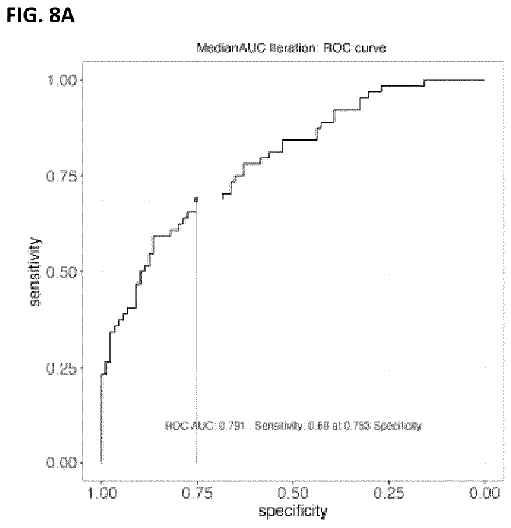

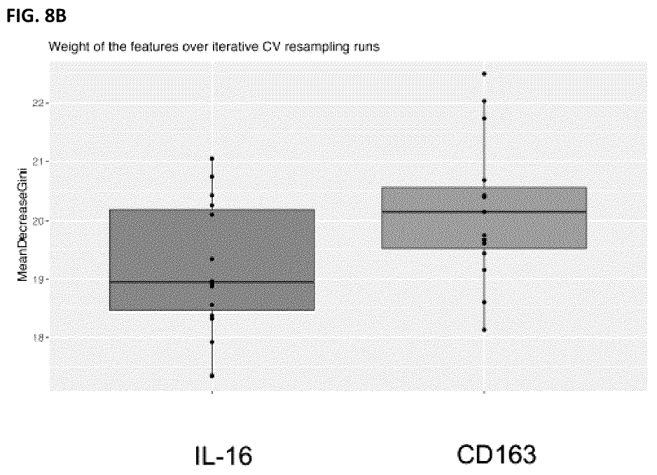

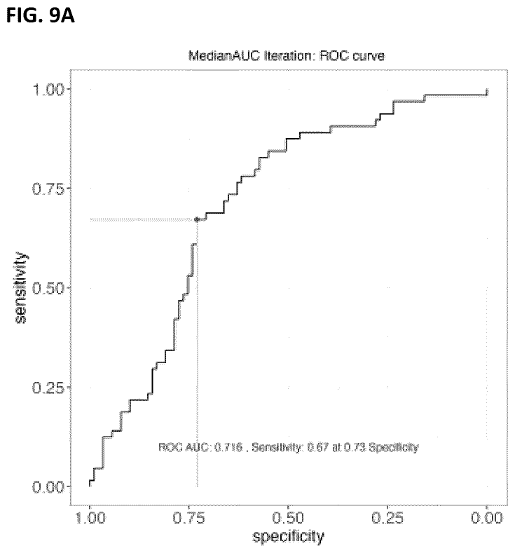

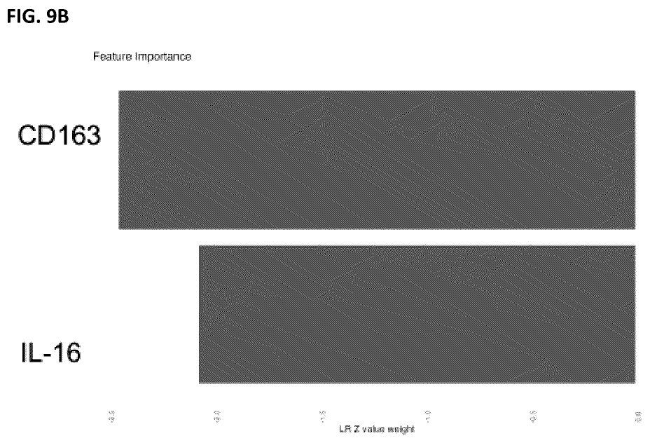

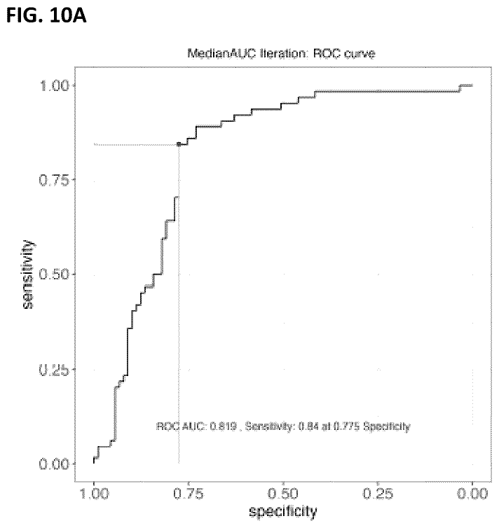

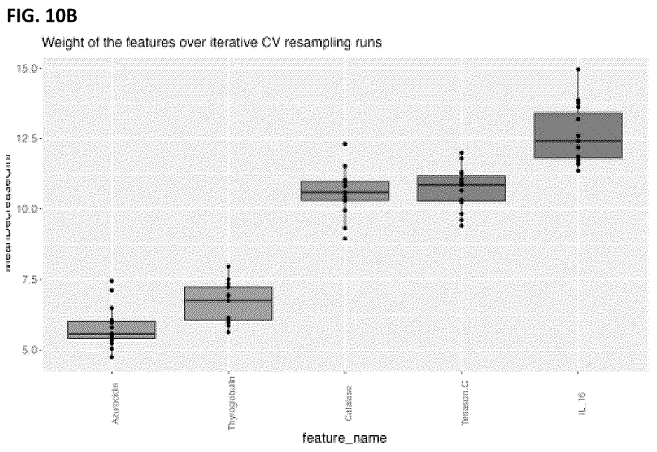

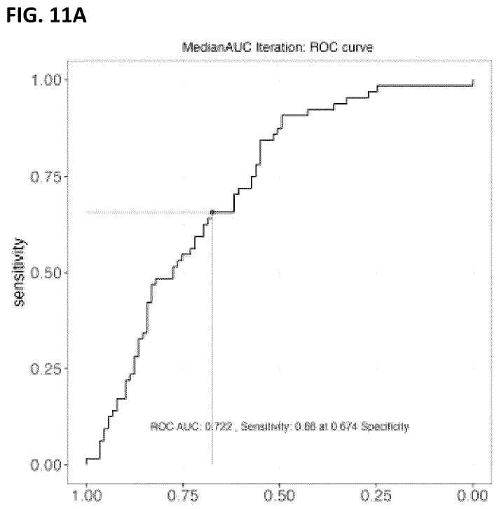

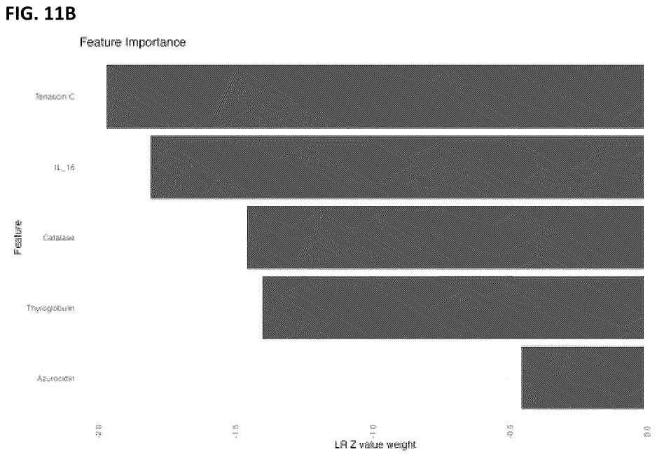

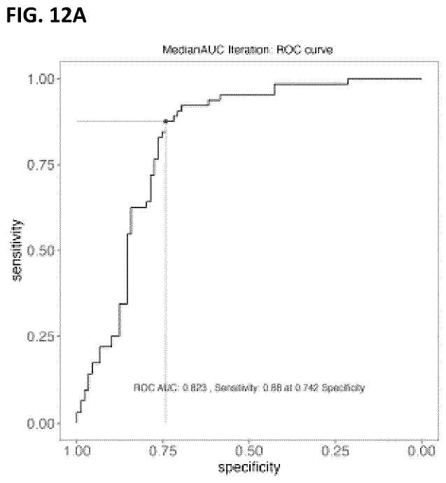

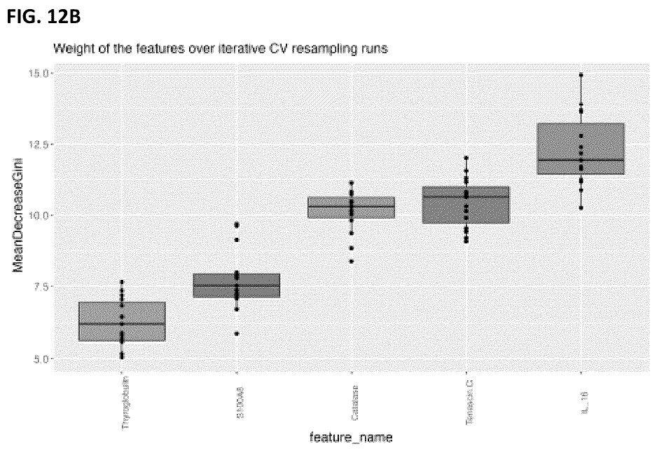

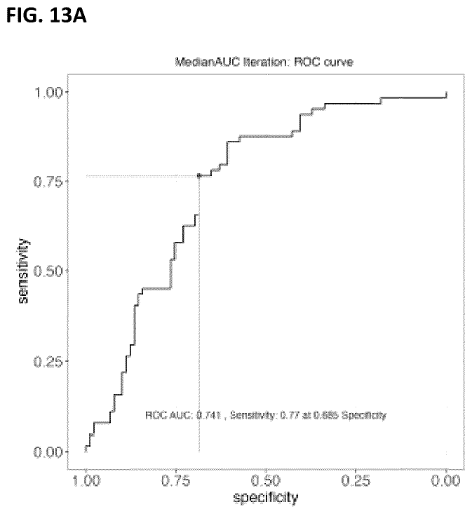

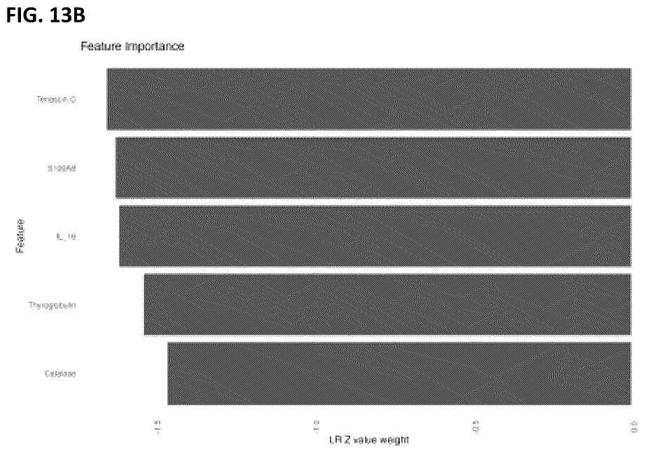

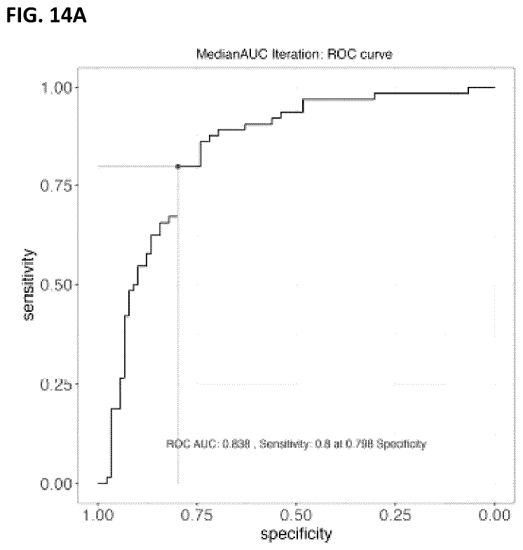

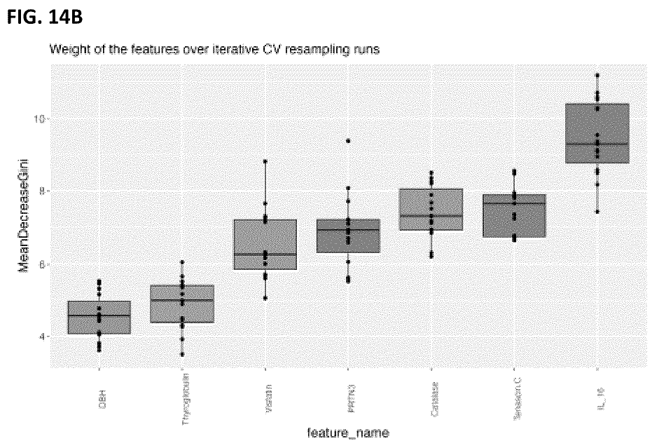

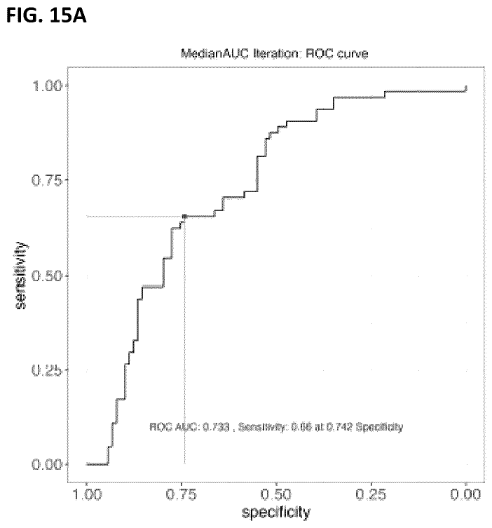

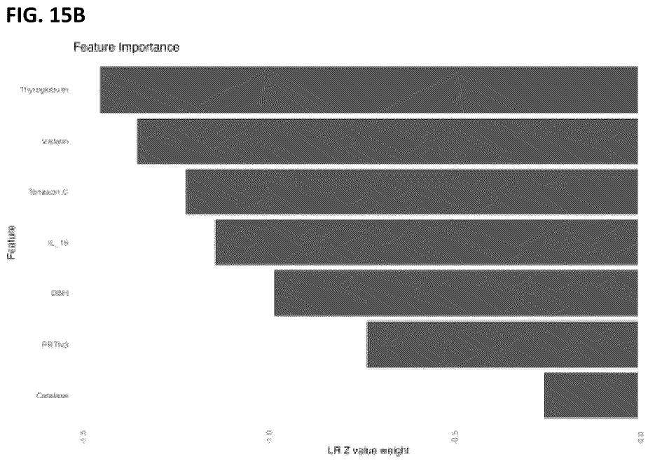

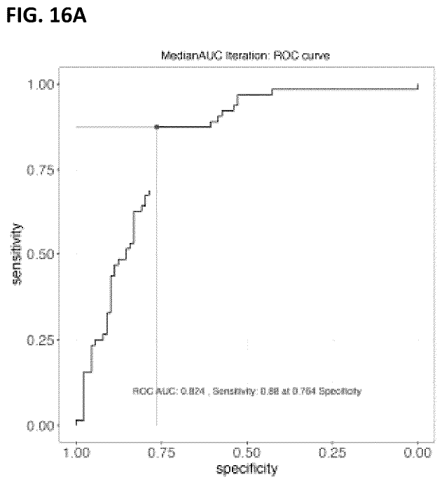

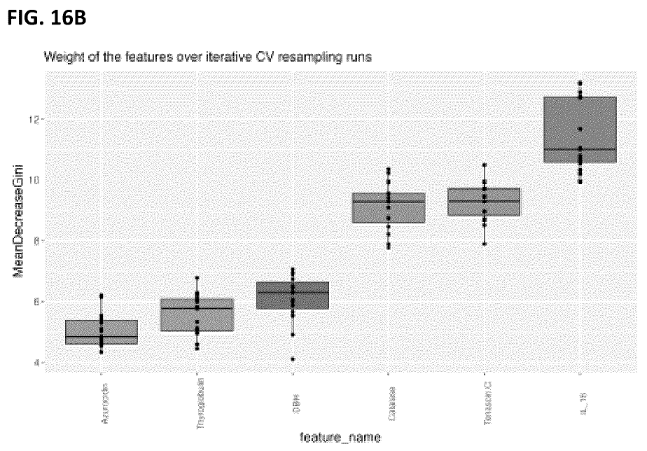

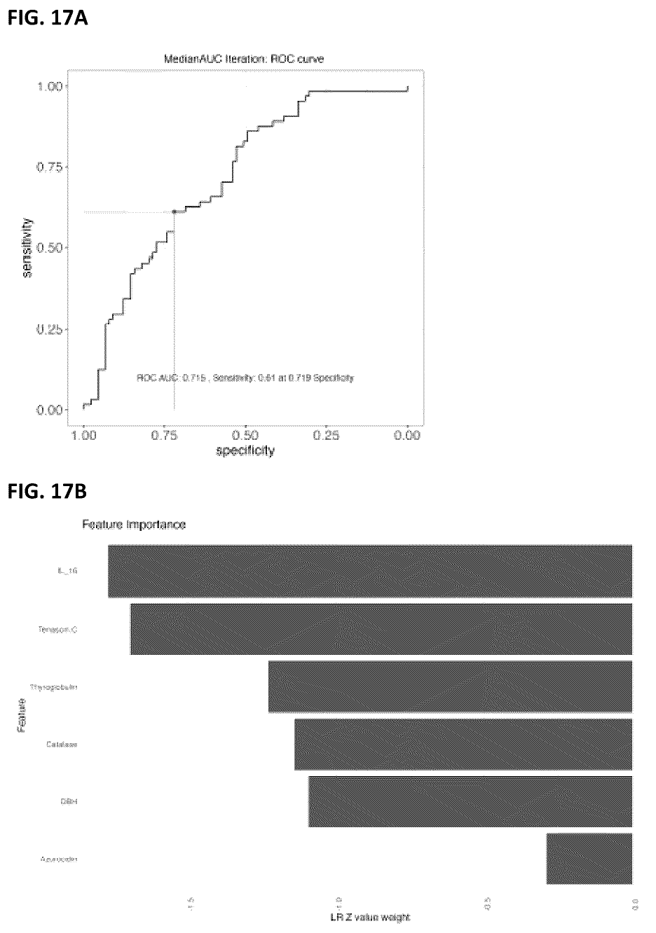

In embodiments of the methods described herein, the proteins further comprise a protein identified in FIG. 6A, a protein identified in FIG. 6B, a protein identified in FIG. 7, or a combination of two or more proteins identified in FIG. 6A and/or FIG. 6B and/or FIG. 7. In embodiments of the methods described herein, the proteins further comprise a protein identified in FIG. 6A, a protein identified in FIG. 6B, or a combination of two or more proteins identified in FIG. 6A and/or FIG. 6B. In embodiments, the proteins further comprise the proteins identified in FIG. 6A and FIG. 6B. In embodiments, the proteins further comprise a protein identified in FIG. 6A or a combination of two or more proteins identified in FIG. 6A. In embodiments, the proteins further comprise the proteins identified in FIG. 6A. In embodiments, the proteins further comprise a protein identified in FIG. 6B or a combination of two or more proteins identified in FIG. 6B. In embodiments, the proteins further comprise the proteins identified in FIG. 6B. In embodiments, the proteins further comprise a protein identified in FIG. 7 or a combination of two or more proteins identified in FIG. 7. In embodiments, the proteins further comprise the proteins identified in FIG. 7.

### Lupus Nephritis

In embodiments of the methods described herein, the lupus nephritis is any type or class of lupus nephritis. In embodiments, the lupus nephritis is histologically active lupus nephritis. In embodiments, the patient has an NIH Activity Index greater than 2. The NIH Activity Index is known in the art and described, for example, by Bajema et al, Kidney Int., 93(4):789-796 (2018) and Choi et al, Kidney Res Clin Pract, 42(2):166-173 (2023), the disclosures of which are incorporated by reference herein in their entirety.

In embodiments of the methods described herein, histologically active is any patient with any class and NIH Activity Index >2. In embodiments, a patient is any patient with class III, IV or mixed with an NIH Index >2. In embodiments, all other classes, regardless of NIH Activity Index are control.

In embodiments of the methods described herein, the lupus nephritis is proliferative lupus nephritis. In embodiments, the lupus nephritis is membranous lupus nephritis. In embodiments, the lupus nephritis is mixed lupus nephritis. In embodiments, the lupus nephritis is Class I lupus nephritis. In embodiments, the lupus nephritis is Class II lupus nephritis. In embodiments, the lupus nephritis is Class III lupus nephritis. In embodiments, the lupus nephritis is Class IV lupus nephritis. In embodiments, the lupus nephritis is Class V lupus nephritis. In embodiments, the lupus nephritis is Class VI lupus nephritis.

In embodiments of the methods described herein, the lupus nephritis is histologically active proliferative lupus nephritis. In embodiments, the lupus nephritis is histologically active membranous lupus nephritis. In embodiments, the lupus nephritis is histologically active mixed lupus nephritis. In embodiments, the lupus nephritis is histologically active Class I lupus nephritis. In embodiments, the lupus nephritis is histologically active Class II lupus nephritis. In embodiments, the lupus nephritis is histologically active Class III lupus nephritis. In embodiments, the lupus nephritis is histologically active Class IV lupus nephritis. In embodiments, the lupus nephritis is histologically active Class V lupus nephritis. In embodiments, the lupus nephritis is histologically active Class VI lupus nephritis.

In embodiments of the methods described herein, the lupus nephritis is proliferative lupus nephritis and the patient has an NIH Activity Index greater than 2. In embodiments, the lupus nephritis is membranous lupus nephritis and the patient has an NIH Activity Index greater than 2. In embodiments, the lupus nephritis is mixed lupus nephritis and the patient has an NIH Activity Index greater than 2. In embodiments, the lupus nephritis is Class I lupus nephritis and the patient has an NIH Activity Index greater than 2. In embodiments, the lupus nephritis is Class II lupus nephritis and the patient has an NIH Activity Index greater than 2. In embodiments, the lupus nephritis is Class III lupus nephritis and the patient has an NIH Activity Index greater than 2. In embodiments, the lupus nephritis is Class IV lupus nephritis and the patient has an NIH Activity Index greater than 2. In embodiments, the lupus nephritis is Class V lupus nephritis and the patient has an NIH Activity Index greater than 2. In embodiments, the lupus nephritis is Class VI lupus nephritis and the patient has an NIH Activity Index greater than 2.

In embodiments of the methods described herein, the lupus nephritis is mixed proliferative lupus nephritis. In embodiments, the lupus nephritis is mixed proliferative Class III and Class IV lupus nephritis. In embodiments, the lupus nephritis is mixed proliferative Class IV and Class V lupus nephritis.

In embodiments of the methods described herein, the lupus nephritis is mixed proliferative Class I lupus nephritis and the patient has an NIH Activity Index greater than 2. In embodiments, the lupus nephritis is mixed proliferative Class II lupus nephritis and the patient has an NIH Activity Index greater than 2. In embodiments, the lupus nephritis is mixed proliferative Class III lupus nephritis and the patient has an NIH Activity Index greater than 2. In embodiments, the lupus nephritis is mixed proliferative Class IV lupus nephritis and the patient has an NIH Activity Index greater than 2. In embodiments, the lupus nephritis is mixed proliferative Class V lupus nephritis and the patient has an NIH Activity Index greater than 2.

In embodiments of the methods described herein, the lupus nephritis is proliferative Class I lupus nephritis. In embodiments, the lupus nephritis is proliferative Class II lupus nephritis. In embodiments, the lupus nephritis is proliferative Class III lupus nephritis. In embodiments, the lupus nephritis is proliferative Class IV lupus nephritis. In embodiments, the lupus nephritis is proliferative Class V lupus nephritis. In embodiments, the lupus nephritis is histologically active proliferative Class I lupus nephritis. In embodiments, the lupus nephritis is histologically active proliferative Class II lupus nephritis. In embodiments, the lupus nephritis is histologically active proliferative Class III lupus nephritis. In embodiments, the lupus nephritis is histologically active proliferative Class IV lupus nephritis. In embodiments, the lupus nephritis is histologically active proliferative Class V lupus nephritis.

In embodiments of the methods described herein, the lupus nephritis is proliferative Class I lupus nephritis and the patient has an NIH Activity Index greater than 2. In embodiments, the lupus nephritis is proliferative Class II lupus nephritis and the patient has an NIH Activity Index greater than 2. In embodiments, the lupus nephritis is proliferative Class III lupus nephritis and the patient has an NIH Activity Index greater than 2. In embodiments, the lupus nephritis is proliferative Class IV lupus nephritis and the patient has an NIH Activity Index greater than 2. In embodiments, the lupus nephritis is proliferative Class V lupus nephritis and the patient has an NIH Activity Index greater than 2.

In embodiments of the methods described herein, the lupus nephritis is membranous lupus nephritis. In embodiments, the lupus nephritis is membranous Class III lupus nephritis. In embodiments, the lupus nephritis is membranous Class IV lupus nephritis. In embodiments, the lupus nephritis is membranous Class V lupus nephritis. In embodiments, the lupus nephritis is histologically active membranous Class III lupus nephritis. In embodiments, the lupus nephritis is histologically active membranous Class IV lupus nephritis. In embodiments, the lupus nephritis is histologically active membranous Class V lupus nephritis.

In embodiments of the methods described herein, the lupus nephritis is membranous Class I lupus nephritis and the patient has an NIH Activity Index greater than 2. In embodiments, the lupus nephritis is membranous Class II lupus nephritis and the patient has an NIH Activity Index greater than 2. In embodiments, the lupus nephritis is membranous Class III lupus nephritis and the patient has an NIH Activity Index greater than 2. In embodiments, the lupus nephritis is membranous Class IV lupus nephritis and the patient has an NIH Activity Index greater than 2. In embodiments, the lupus nephritis is membranous Class V lupus nephritis and the patient has an NIH Activity Index greater than 2.

In embodiments of the methods described herein, the methods further comprise detecting: (a) an elevated level of complement component 3 (C3) in the biological sample obtained from the subject; (b) an elevated level of complement component 4 (C4) in the biological sample obtained from the subject; (c) an elevated level of anti-dsDNA in the biological sample obtained from the subject; (d) an elevated level of proteinuria in the biological sample obtained from the subject; or (e) a combination of two or more of (a), (b), (c), and (d). In embodiments, the elevated level is relative to a control (as described herein). In embodiments, the methods further comprise detecting an elevated level of complement component 3 (C3) in the biological sample obtained from the subject. In embodiments, the methods further comprise detecting an elevated level of complement component 4 (C4) in the biological sample obtained from the subject. In embodiments, the methods further comprise detecting an elevated level of complement component 3 (C3) and an elevated level of complement component 4 (C4) in the biological sample obtained from the subject. In embodiments, the methods further comprise detecting an elevated level of anti-dsDNA in the biological sample obtained from the subject. In embodiments, the methods further comprise detecting an elevated level of complement component 3 (C3) and an elevated level of anti-dsDNA in the biological sample obtained from the subject. In embodiments, the methods further comprise detecting an elevated level of complement component 4 (C4), and an elevated level of anti-dsDNA in the biological sample obtained from the subject. In embodiments, the methods further comprise detecting an elevated level of complement component 3 (C3), an elevated level of complement component 4 (C4), and an elevated level of anti-dsDNA in the biological sample obtained from the subject. In embodiments, the methods further comprise detecting an elevated level of proteinuria in the biological sample obtained from the subject. In embodiments, the methods further comprise detecting an elevated level of complement component 3 (C3), an elevated level of complement component 4 (C4), an elevated level of anti-dsDNA, and an elevated level of proteinuria in the biological sample obtained from the subject.

### Biological Sample

“Biological sample” or “sample” refer to materials obtained from a patient. A biological sample includes tissue, cells, and bodily fluids such as blood and blood fractions or products (e.g., serum, plasma, platelets, red blood cells), sputum, tissue, cultured cells (e.g., primary cultures, explants, transformed cells), stool, urine, sweat, saliva, synovial fluid, joint tissue, synovial tissue, immune cells, hematopoietic cells, macrophages, and the like. In embodiments of the methods described herein, a biological sample is blood (e.g., alternatively referred to as a blood sample). In embodiments, a biological sample is a serum sample. In embodiments, a biological sample is a plasma sample.

In embodiments of the methods described herein, a biological sample is urine (e.g., a urine sample).

### Lupus Nephritis Therapeutic Agent

The methods described herein include administering to a patient an effective amount of a lupus nephritis therapeutic agent. In embodiments, the lupus nephritis therapeutic agent is an immunosuppressive agent, a B-cell inhibitor, an interferon antagonist, a glucocorticoid, or a combination of two or more thereof.

In embodiments of the methods described herein, the lupus nephritis therapeutic agent is an immunosuppressive agent. In embodiments, the immunosuppressive agent is methotrexate, leflunomide, hydroxychloroquine, azathioprine, cyclosporine, cyclophosphamide, mycophenolate, voclosporin, or a combination of two or more thereof. In embodiments, the immunosuppressive agent is methotrexate. In embodiments, the immunosuppressive agent is leflunomide. In embodiments, the immunosuppressive agent is hydroxychloroquine. In embodiments, the immunosuppressive agent is azathioprine. In embodiments, the immunosuppressive agent is cyclosporine. In embodiments, the immunosuppressive agent is cyclophosphamide. In embodiments, the immunosuppressive agent is mycophenolate. In embodiments, the immunosuppressive agent is voclosporin.

In embodiments of the methods described herein, the lupus nephritis therapeutic agent is a B-cell inhibitor. In embodiments, the B-cell inhibitor is belimumab, rituximab, ocrelizumab, veltuzumab, obinutuzumab, ibritumomab tiuxetan, ofatumumab, or epratuzumab. In embodiments, the B-cell inhibitor is belimumab. In embodiments, the B-cell inhibitor is rituximab. In embodiments, the B-cell inhibitor is ocrelizumab. In embodiments, the B-cell inhibitor is veltuzumab. In embodiments, the B-cell inhibitor is obinutuzumab. In embodiments, the B-cell inhibitor is ibritumomab tiuxetan. In embodiments, the B-cell inhibitor is ofatumumab. In embodiments, the B-cell inhibitor is epratuzumab.

In embodiments of the methods described herein, the lupus nephritis therapeutic agent is an interferon antagonist. In embodiments, the interferon antagonist is anifrolumab, IFN-α kinoid, rontalizumab, sifalimumab, or bortezomib. In embodiments, the interferon antagonist is anifrolumab. In embodiments, the interferon antagonist is IFN-α kinoid. In embodiments, the interferon antagonist is rontalizumab. In embodiments, the interferon antagonist is sifalimumab. In embodiments, the interferon antagonist is bortezomib.

In embodiments of the methods described herein, the lupus nephritis therapeutic agent is a glucocorticoid. In embodiments, the glucocorticoid is prednisone, methylprednisolone, dexamethasone, or betamethasone. In embodiments, the glucocorticoid is prednisone. In embodiments, the glucocorticoid is methylprednisolone. In embodiments, the glucocorticoid is dexamethasone. In embodiments, the glucocorticoid is betamethasone.

### Kits and Assays

Provided here are kits comprising components, such as reagents and reaction mixtures, to conduct the assays to detect proteins comprising CEACAM-1, CD163, IP-10, C5a, P-cadherin, IL-16, cathepsin S, PRX1, NCAM-1, catalase, FOLR2, PRTN3, or a combination of two or more thereof, and optionally further comprising cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, SULT1B1, or a combination of two or more thereof, as described herein. As part of the kit, materials and instruction are provided, e.g., for storage and use of kit components. In embodiments, the proteins further comprise ANG-4, B7-2, BAMBI, BLMH, ClqTNF1, CD155, CD229, CD27, CD40L, calsyntenin-1, caspase 7, CXADR, Cf10, contactin-4, cripto-1, DCTN1, DLL1, DR6, Dkk-3, ephrin-B3, FABP6, FGF-5, GFR alpha-2, glypican 2, hepassocin, IL-13 R2, IL-22, IL-23, LDL R, LRRC4, legumain, MBL, MCP-2, MIP-3b, MMP-1, melan-A, nectin-1, neuropilin-1, PCDH17, PTH, Pref-1, SLPI, Shh-N, siglec-5, stabilin-2, TFF3, TIM-1, thrombospondin-5, aFGF, angiostatin, C-myc, FGF-20, GDF-11, GITR L, PCK1, RGM-C, thrombospondin-2, ZAP70, or a combination of two or more thereof. In embodiments, the biomarkers comprise CEACAM-1, CD163, IP-10, C5a, P-cadherin, IL-16, cathepsin S, PRX1, NCAM-1, catalase, FOLR2, and PRTN3. In embodiments, the biomarkers consist of CEACAM-1, CD163, IP-10, C5a, P-cadherin, IL-16, cathepsin S, PRX1, NCAM-1, catalase, FOLR2, and PRTN3. In embodiments, the biomarkers comprise CEACAM-1, CD163, IP-10, C5a, P-cadherin, IL-16, cathepsin S, PRX1, NCAM-1, catalase, FOLR2, PRTN3, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, SULT1B1. In embodiments, the biomarkers consist of CEACAM-1, CD163, IP-10, C5a, P-cadherin, IL-16, cathepsin S, PRX1, NCAM-1, catalase, FOLR2, PRTN3, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, and SULT1B1.

In embodiments, the kit comprises a plurality of antibodies attached to a solid support wherein the antibodies bind to a plurality of proteins comprising CEACAM-1, CD163, IP-10, C5a, P-cadherin, IL-16, cathepsin S, PRX1, NCAM-1, catalase, FOLR2, PRTN3, or a combination of two or more thereof, and optionally further comprising cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, SULT1B1, or a combination of two or more thereof, as described herein. In embodiments, the proteins further comprise ANG-4, B7-2, BAMBI, BLMH, ClqTNF1, CD155, CD229, CD27, CD40L, calsyntenin-1, caspase 7, CXADR, Cf10, contactin-4, cripto-1, DCTN1, DLL1, DR6, Dkk-3, ephrin-B3, FABP6, FGF-5, GFR alpha-2, glypican 2, hepassocin, IL-13 R2, IL-22, IL-23, LDL R, LRRC4, legumain, MBL, MCP-2, MIP-3b, MMP-1, melan-A, nectin-1, neuropilin-1, PCDH17, PTH, Pref-1, SLPI, Shh-N, siglec-5, stabilin-2, TFF3, TIM-1, thrombospondin-5, aFGF, angiostatin, C-myc, FGF-20, GDF-11, GITR L, PCK1, RGM-C, thrombospondin-2, ZAP70, or a combination of two or more thereof. In embodiments, the protein comprises CEACAM-1, CD163, IP-10, C5a, P-cadherin, IL-16, cathepsin S, PRX1, NCAM-1, catalase, FOLR2, and PRTN3. In embodiments, the proteins consist of CEACAM-1, CD163, IP-10, C5a, P-cadherin, IL-16, cathepsin S, PRX1, NCAM-1, catalase, FOLR2, and PRTN3. In embodiments, the protein comprises CEACAM-1, CD163, IP-10, C5a, P-cadherin, IL-16, cathepsin S, PRX1, NCAM-1, catalase, FOLR2, PRTN3, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, SULT1B1. In embodiments, the proteins consist of CEACAM-1, CD163, IP-10, C5a, P-cadherin, IL-16, cathepsin S, PRX1, NCAM-1, catalase, FOLR2, PRTN3, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, IFN-gamma R1, HNMT, galectin-4, fetuin B, CRTAM, MFRP, SULT1B1.

Provided here are kits comprising components, such as reagents and reaction mixtures, to conduct the assays to detect proteins comprising CD163, IL-16, azurocidin, tenascin C, IL-16, catalase, thyroglobulin, S100A8, PRTN3, DBH, visfatin, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, or a combination of two or more thereof, as described herein. As part of the kit, materials and instruction are provided, e.g., for storage and use of kit components. The protein comprises or consist of any proteins described herein, whether they are individual proteins or any combination of two or more proteins. Descriptions of the individual proteins and exemplary combinations of the proteins are described in detail herein.

In embodiments, the kit comprises a plurality of antibodies attached to a solid support wherein the antibodies bind to a plurality of proteins comprising CD163, IL-16, azurocidin, tenascin C, IL-16, catalase, thyroglobulin, S100A8, PRTN3, DBH, visfatin, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, or a combination of two or more thereof, as described herein. As part of the kit, materials and instruction are provided, e.g., for storage and use of kit components. The protein comprises or consist of any proteins described herein, whether they are individual proteins or any combination of two or more proteins. Descriptions of the individual proteins and exemplary combinations of the proteins are described in detail herein.

“Assaying” or “detecting” means using an analytic procedure to qualitatively assess or quantitatively measure the presence or amount or the functional activity of a target entity (e.g., protein levels). For example, detecting the level of a protein means using an analytic procedure (such as an in vitro procedure) to qualitatively assess or quantitatively measure the presence or amount of the protein.

The terms “probe” or “primer” refer to one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe or primers can be unlabeled or labeled as described below so that its binding to a target sequence can be detected (e.g., with a FRET donor or acceptor label). The probe or primer can be designed based on one or more particular (preselected) portions of a chromosome, e.g., one or more clones, an isolated whole chromosome or chromosome fragment, or a collection of polymerase chain reaction (PCR) amplification products. One of skill can adjust these factors to provide optimum hybridization and signal production for a given hybridization and detection procedures, and to provide the required resolution among different genes or genomic locations. Probes and primers can also be immobilized on a solid surface (e.g., nitrocellulose, glass, quartz, fused silica slides), as in an array. Techniques for producing high density arrays can also be used. One of skill will recognize that the precise sequence of particular probes and primers can be modified from the target sequence to a certain degree to produce probes that are substantially identical or substantially complementary to a target sequence, but retain the ability to specifically bind to (i.e., hybridize specifically to) the same targets from which they were derived.

In embodiments, methods include detecting a level of a biomarker with a specific binding agent (e.g., an agent that binds to a protein or nucleic acid molecule). Exemplary specific binding agents include an antibody or a fragment thereof, a detectable protein or a fragment thereof, a nucleic acid molecule such as an oligonucleotide/polynucleotide comprising a sequence that is complementary to patient genomic DNA, mRNA or a cDNA produced from patient mRNA, or any combination thereof. In embodiments, an antibody is labeled with detectable moiety, e.g., a fluorescent compound, an enzyme or functional fragment thereof, or a radioactive agent. In embodiments, an antibody is detectably labeled by coupling it to a chemiluminescent compound. In embodiments, the presence of the chemiluminescent-tagged antibody is then determined by detecting the presence of luminescence that arises during the course of chemical reaction.

In embodiments, a specific binding agent is an agent that has greater than 10-fold, preferably greater than 100-fold, and most preferably, greater than 1000-fold affinity for the target molecule as compared to another molecule. As the skilled artisan will appreciate the term specific is used to indicate that other biomarkers present in the sample do not significantly bind to the binding agent specific for the target molecule. In embodiments, the level of binding to a biomolecule other than the target biomarker results in a binding affinity which is at most only 10% or less, only 5% or less only 2% or less or only 1% or less of the affinity to the target molecule, respectively. A preferred specific binding agent will fulfill both the above minimum criteria for affinity as well as for specificity. For example, in embodiments an antibody has a binding affinity (e.g., Kd) in the low micromolar (10⁻⁶), nanomolar (10⁻⁷-10⁻⁹), with high affinity antibodies in the low nanomolar (10⁻⁹) or pico molar (10⁻¹²) range for its specific target biomarker.

In embodiments, the kit comprises a specific binding agent, wherein the specific binding agent is attached to a solid support, (e.g., a strip, a polymer, a bead, a nanoparticle, a plate such as a multiwell plate, or an array such as a microarray). In embodiments, the specific binding agent is an antibody. In embodiments relating to the use of a nucleic acid probe attached to a solid support (such as a microarray), a nucleic acid in a test sample may be amplified (e.g., using PCR) before or after the nucleic acid to be measured is hybridized with the probe. In embodiments, reverse transcription polymerase chain reaction (RT-PCR) is used to detect mRNA levels. In embodiments, the support or carrier comprises glass, polystyrene, polypropylene, polyethylene, dextran, nylon, amylases, natural and modified celluloses, polyacrylamides, and magnetite. In embodiments, the nature of the carrier can be either soluble to some extent or insoluble for the purposes of the present subject matter. In embodiments, the support material is any structural configuration so long as the coupled molecule is capable of binding to a binding agent (e.g., an antibody). In embodiments, the support configuration may be spherical, as in a bead, or cylindrical, as in the inside surface of a test tube, or the external surface of a rod. In embodiments, the surface may be flat such as a plate (or a well within a multiwell plate), sheet, test strip, polystyrene beads. The skilled artisan will know other suitable carriers for binding antibody or antigen or will be able to ascertain the same by use of routine experimentation.

In embodiments, a solid support comprises a polymer, to which an agent is chemically bound, immobilized, dispersed, or associated. In embodiments, a polymer support may be, e.g., a network of polymers, and may be prepared in bead form (e.g., by suspension polymerization). In embodiments, the location of active sites introduced into a polymer support depends on the type of polymer support. In embodiments, in a swollen-gel-bead polymer support the active sites are distributed uniformly throughout the beads, whereas in a macroporous-bead polymer support they are predominantly on the internal surfaces of the macropores. In embodiments, the solid support, e.g., a device, may contain a biomarker binding agent alone or together with a binding agent for at least one, two, three or more other biomarkers.

In embodiments, the level of a protein are determined by an immunoassay, liquid chromatography-mass spectrometry (LC-MS), ELISA, or a combination thereof. In embodiments, the level of a protein are determined by an immunoassay. In embodiments, the level of a protein are determined by an liquid chromatography-mass spectrometry. In embodiments, the level of a protein are determined by ELISA. In embodiments, the ELISA is multiplex ELISA.

In embodiments of the methods described herein, the methods further comprise calculating a lupus nephritis score. In embodiments, calculating a lupus nephritis score comprises adding the relative levels of the proteins. In embodiments, the relative level of the protein is the log 2 relative level of the protein. In embodiments, the level of a protein is normalized to the level of creatinine.

In embodiments, the lupus nephritis score is the median of the sum of the relative levels of the four genes. In embodiments, the relative level of each gene is the level of each gene divided by the median normalized level of the gene from a control. In embodiments, a positive lupus nephritis score indicates that the patient has lupus nephritis. In embodiments, a positive lupus nephritis score indicates that the patient with SLE is at risk of developing lupus nephritis. In embodiments, a negative score indicates that the patient does not have lupus nephritis. In embodiments, a negative score indicates that the patient with SLE is not at risk of developing lupus nephritis.

### EXAMPLES

### Example 1

Achieving an NIH Activity Index of ≤2 upon treatment is associated with less lupus nephritis flare and thus better long-term kidney survival. Here, we developed and validated a panel of urinary protein biomarkers to predict an NIH Activity Index >2.

A panel of 1,200 proteins (Kiloplex, RayBiotech) was measured in urine samples collected within 3 weeks of kidney biopsy and at weeks 12, 24 and 52 in lupus nephritis patients by the Accelerating Medicines Partnership in RA/SLE (publicly available). The classifier was trained to identify proliferative lupus nephritis with an NIH Activity Index >2. A series of machine learning classifiers were tested to identify the performing model in terms of AUC and LogLoss by cross validation in 80% of the data. Model performance was validated in a distinct 20% partition of the data. Renal response was defined at one year from biopsy in patients with baseline UPCR≥1: Complete=UPCR<0.5, serum creatinine <125% of baseline, prednisone ≤10 mg/d; Partial=UPCR<50% from baseline but >0.5, serum creatine <125% of baseline, but prednisone up to 15 mg/d; Non-responders did not meet prior definitions.

Assay Performance/Calibration Curve Data+Patient Data Overlay. To refine this selection, a multi-faceted approach was employed to analyze the immunoassay quality of each candidate. This process involved a detailed examination of the standard curves for each protein using raw response data. Subsequently, raw response values from 596 patient samples were overlaid onto each protein's individual standard curve. This overlay allowed for a comprehensive assessment of how patient sample values are distributed across each protein's standard curve, providing crucial context for the multiplex data. By understanding the distribution of raw patient response values and their positioning on the standard curves, we could evaluate the reliability and trustworthiness of the multiplex data for each candidate protein. This meticulous approach ensured that the final selection of biomarkers was not only clinically informative but also analytically robust, thereby providing a solid foundation for candidate selection. The process effectively whittled down the initial subset of candidates to a refined list of biomarkers with verified analytical performance within the context of actual patient samples. Importantly, when a candidate protein's calibration curve characteristics were found to be of insufficient quality for patient value overlay, it prompted a search for a suitable replacement, ensuring that only proteins with robust analytical performance were retained in the final biomarker panels.

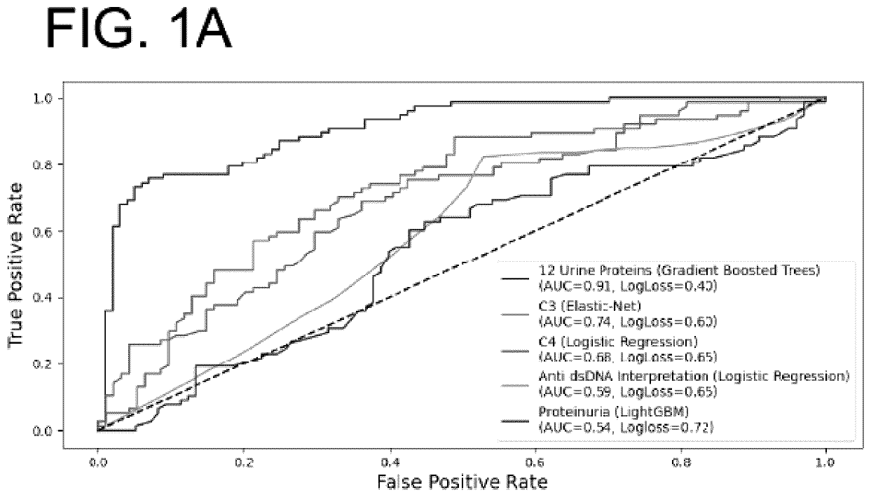

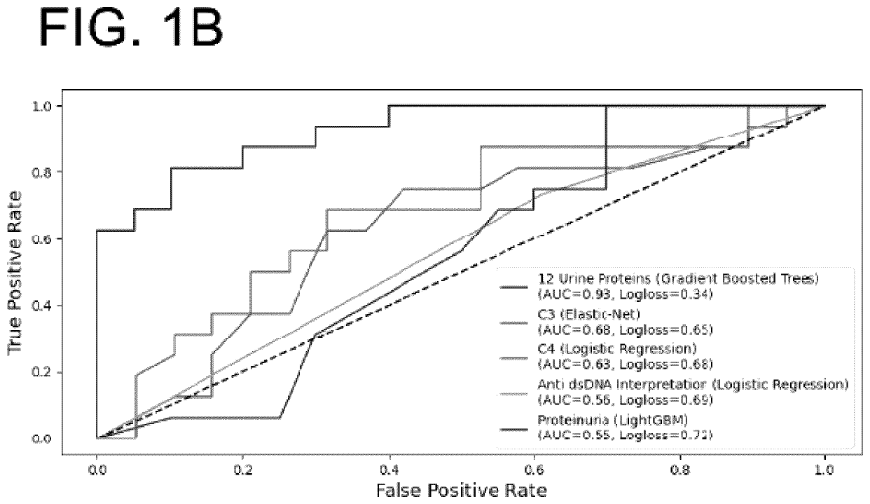

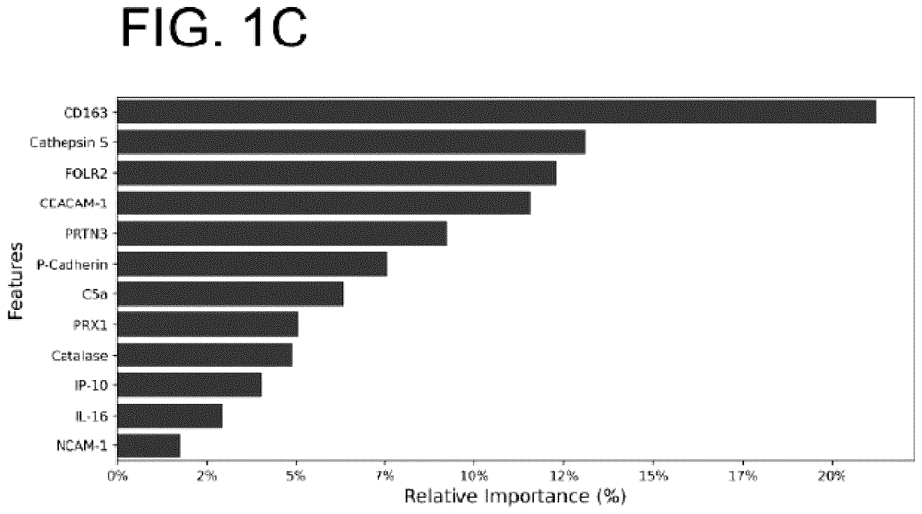

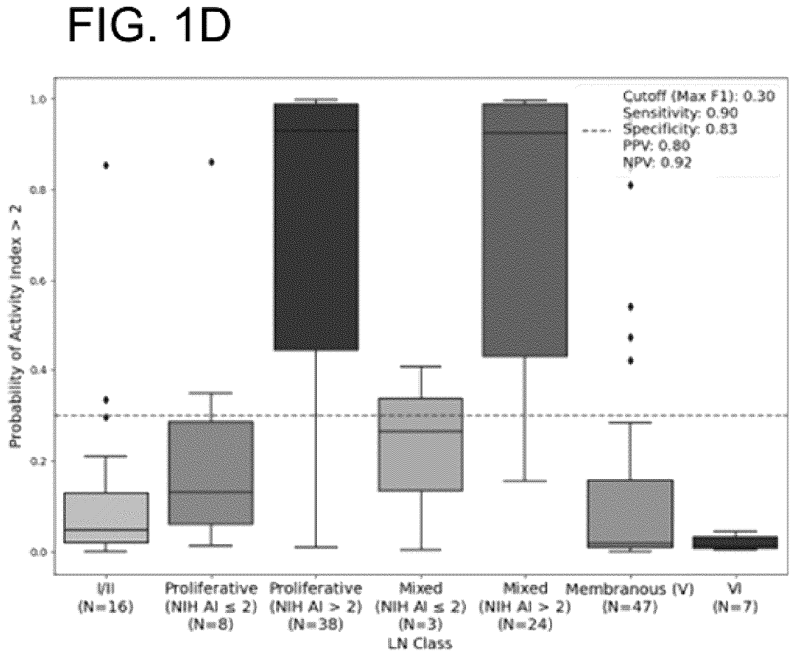

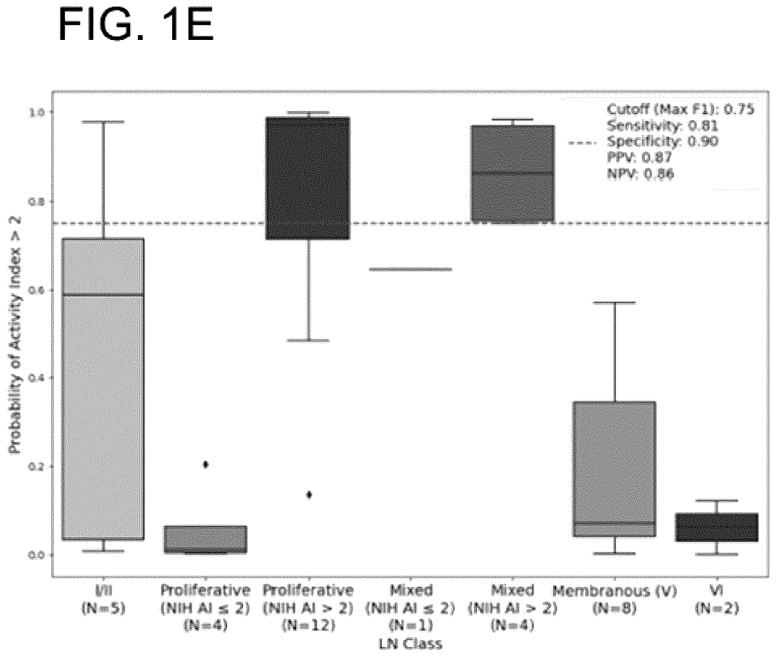

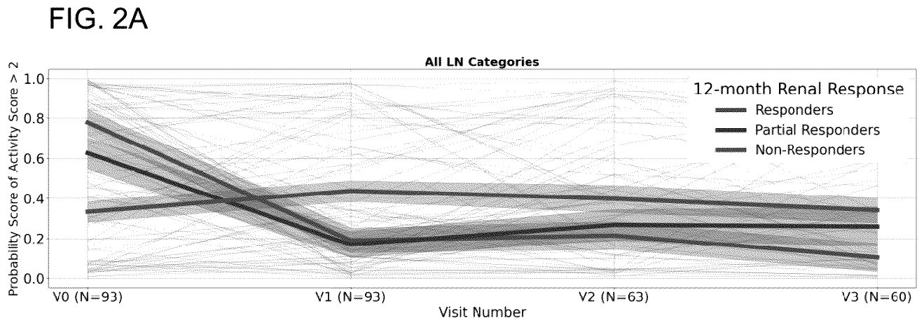

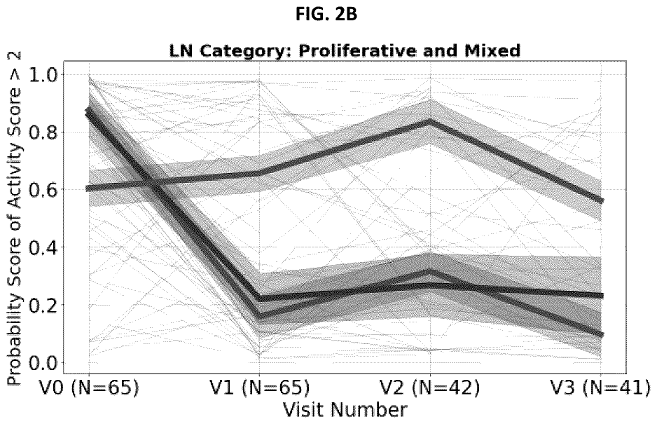

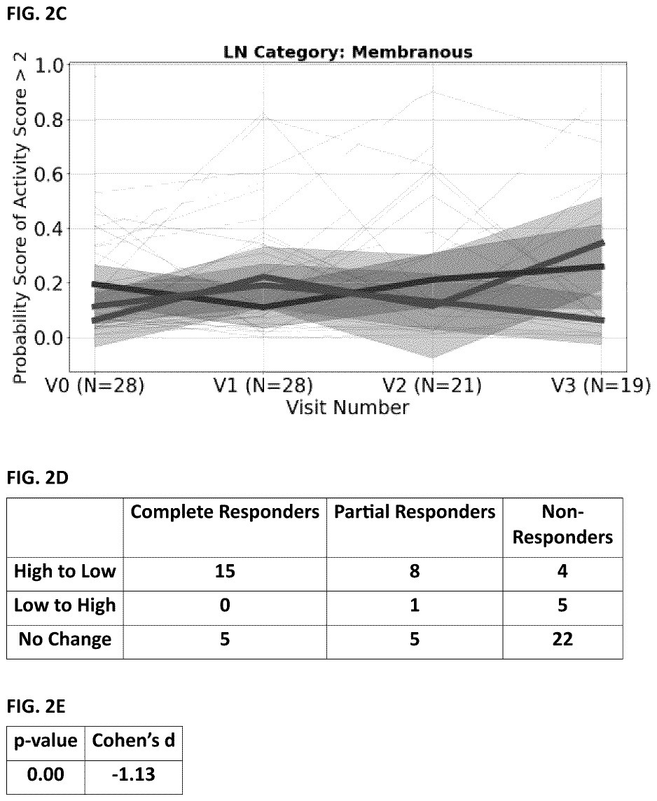

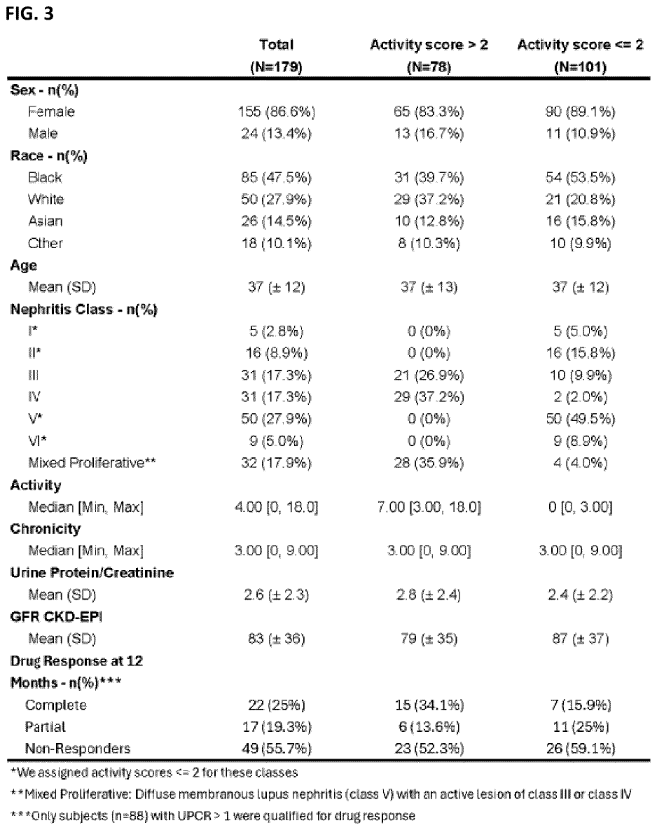

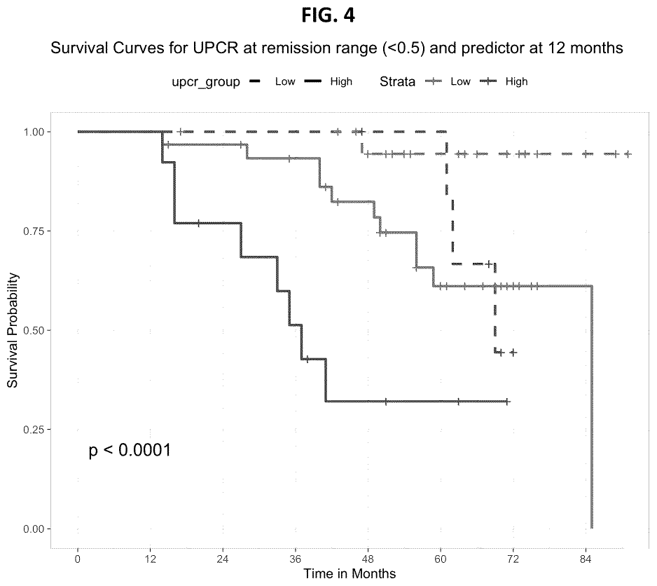

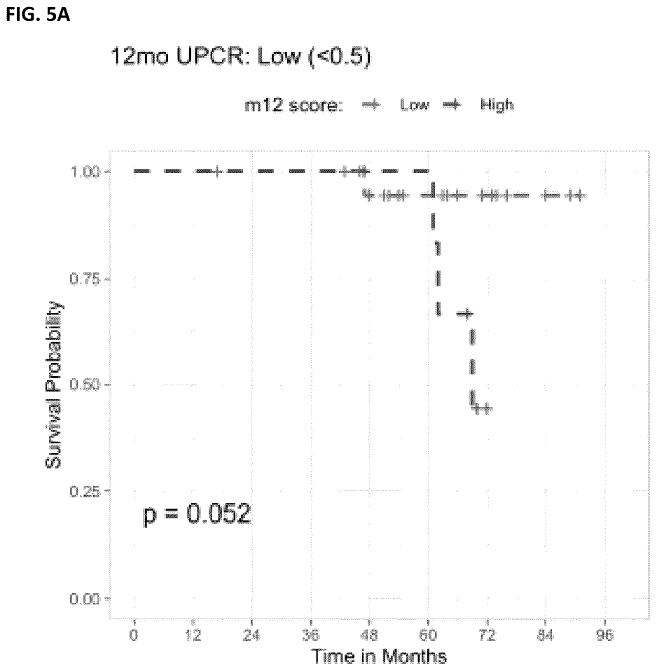

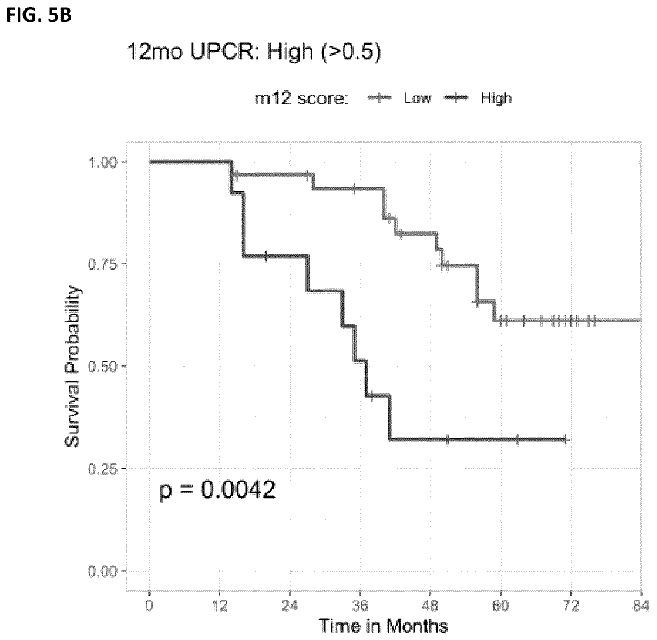

As shown in FIG. 3, a cohort of 179 subjects were included with proliferative lupus nephritis, membranous lupus nephritis, and mixed lupus nephritis (FIG. 3). We identified a panel of 12 urinary proteins that maximized the performance to robustly predict an NIH Activity Index >2 (AUC 90%) while minimizing the number of biomarkers (FIG. 1A). This gradient boosted tree classifier model predicted an NIH Activity Index >2 with an AUC of 90% for the cross-validation outperforming clinically available biomarkers such as C3 (73%), C4 (67%), anti-dsDNA (60%) and UPCR (59%) (FIG. 1A). We validated the model in a testing set confirming its accuracy (AUC 93%) and improved performance over the clinical biomarkers (FIG. 1B). The top 3 features in terms of relative importance were CD163, cathepsin S, and FOLR2 (FIG. 1C). Because the rapid resolution of histological activity is critical to prevent irreversible kidney damage in lupus nephritis, we studied the trajectories of the biomarker panel as a means to noninvasively track changes in histological activity in response to treatment. Patients who ultimately were classified as complete responders at 1 year displayed a different trajectory of the activity score predictions (FIG. 2A). At the 3-month follow up visit (V1) compared to baseline (V0), proliferative lupus nephritis complete responders had a lower probability of an activity score >2 compared to partial/non-responders (Cohen's d: −1.13, p=0.00) indicating a lower probability of an activity score >2 (FIG. 2B).

TABLE 1

| | UPCR | Anti-dsDNA | C3 | 12 Protein Panel |
|---|---|---|---|---|
| Sensitivity | 100% | 50% | 39% | 81% |
| Specificity | 0% | 7% | 19% | 90% |
| Pos Pred Val | 53% | 33% | 35% | 87% |
| Neg Pred Val | 0% | 13% | 21% | 86% |
| Accuracy | 53% | 28% | 29% | 86% |

We identified a noninvasive urinary biomarker panel that robustly predicted meaningful and actionable histological findings (active proliferative lupus nephritis). It outperformed currently clinical available biomarkers. In contrast to proteinuria, which nonspecifically reflects renal disease, this panel for histological activity includes a set of 12 proteins (i.e., CEACAM-1, CD163, IP-10, C5a, P-cadherin, IL-16, cathepsin S, PRX1, NCAM-1, catalase, FOLR2, PRTN3) linked to intrarenal inflammation. An improvement in the biomarker panel at 3 months predicted a clinical response at 1 year. This biomarker panel will aid in the diagnosis of lupus nephritis and longitudinally guide treatment decisions in lupus nephritis.

### Example 2

Additional biomarkers were identified in humans with lupus nephritis (FIG. 3). Table 2 contains p-values from Mann-Whitney U tests comparing high-activity patients (NIH Activity Index greater than 2) and low-activity patients (NHI Activity Index ≤2). Out of the 28 biomarkers tested, 22 showed significant p-values, confirming their association with disease activity. Six biomarkers were not significant, i.e., IFN-gamma R1, HNMT, CRTAM, fetuin B, SULTIB1, and C1qR1.

TABLE 2

| Protein Biomarker | p value |
|---|---|
| IFN-gamma R1 | 0.84 |
| HNMT | 0.72 |
| CRTAM | 0.72 |
| Fetuin B | 0.41 |
| SULT1B1 | 0.35 |
| C1q R1 | 0.13 |
| Nidogen-1 | 0.06 |
| CXCL16 | 0.06 |
| PCSK9 | 0.03 |
| Cyclophilin A | 0.03 |
| CrkL | 0.03 |
| PAM | 0.02 |
| Galectin-8 | 0.02 |
| ADAM8 | 0.02 |
| MFRP | 0.01 |
| FKBP51 | 0.00 |
| Nestin | 0.00 |
| IL-6 | 0.00 |
| MMR | 0.00 |
| S100A8 | 0.00 |
| CES1 | 0.00 |
| S100A13 | 0.00 |
| Galectin-4 | 0.00 |
| Midkine | 0.00 |
| MIP-1b | 0.00 |
| Tenascin C | 0.00 |
| Galectin-1 | 0.00 |

### Example 3

A model was created with the 6 protein biomarkers in Table 2 that did not show significant p values (i.e., IFN-gamma R1, HNMT, CRTAM, fetuin B, SULTIB1, and C1qR1), following the same methodology used for the 12 protein biomarkers (i.e., CEACAM-1, CD163, IP-10, C5a, P-cadherin, IL-16, cathepsin S, PRX1, NCAM-1, catalase, FOLR2, PRTN3 (12 features)). Although the individual performance of these six biomarkers was not as strong, they will likely provide value when used in specific combinations.

TABLE 3

| | CV | CV | Holdout | Holdout |
|---|---|---|---|---|
| Model | AUC | LogLoss | AUC | LogLoss |
| 12 features and IFN-gamma R1 | 87% | 55% | 87% | 47% |
| 12 features and HNMT | 87% | 54% | 89% | 43% |
| 12 features and CRTAM | 89% | 49% | 90% | 41% |
| 12 features and Fetuin B | 89% | 51% | 88% | 49% |
| 12 features and SULT1B1 | 88% | 52% | 88% | 47% |
| 12 features and C1q R1 | 88% | 50% | 89% | 47% |

### Example 4

Long-term kidney function in humans with lupus nephritis (FIG. 3) was predicted based on protein biomarkers, as shown in Table 4, which shows baseline values for those with 40% or greater GFR loss at 4 years vs. those without; six-month values for those with 40% or greater GFR loss at 4 years vs. those without; twelve-month values for those with 40% GFR loss at 4 years vs. those without; baseline values for those with 30% or greater GFR loss at 3 years vs. those without; six-month values for those with 30% or greater GFR loss at 3 years vs. those without; twelve-month values for those with 30% or greater GFR loss at 3 years vs. those without. The p-values for these 58 protein biomarkers s are significant in at least one scenario. Additional protein biomarkers were identified for predicting long-term kidney function and are in shown in FIGS. 6A-6B and 7.

TABLE 4
p-values from Mann-Whitney U test

| | p value | p value | p value | p value | p value | p value |
|---|---|---|---|---|---|---|
| | 12 month | 6 month | baseline | 12 month | 6 month | baseline |
| Protein | data | Data | data | Data | Data | data |
| Biomarker | 4 years | 4 years | 4 years | 3 years | 3 years | 3 years |
| Thrombospondin-5 | 0.11 | 0.03 | 0.11 | 0.94 | 0.75 | 0.94 |
| MMP-1 | 0.12 | 0 | 0.12 | 0.91 | 0.11 | 0.91 |
| Melan-A | 0.09 | 0.02 | 0.09 | 0.91 | 0.86 | 0.91 |
| FGF-5 | 0.04 | 0.06 | 0.04 | 0.86 | 0.84 | 0.86 |
| Glypican 2 | 0.07 | 0.12 | 0.07 | 0.77 | 0.74 | 0.77 |
| C-myc | 0.28 | 0.03 | 0.28 | 0.67 | 0.11 | 0.67 |
| Stabilin-2 | 0.08 | 0.71 | 0.08 | 0.38 | 0.84 | 0.38 |
| PTH | 0.07 | 0.39 | 0.07 | 0.36 | 0.68 | 0.36 |
| ZAP70 | 0.05 | 0.16 | 0.05 | 0.33 | 0.73 | 0.33 |
| B7-2 | 0.01 | 0.03 | 0.01 | 0.19 | 0.19 | 0.19 |
| CD27 | 0.02 | 0.03 | 0.02 | 0.18 | 0.1 | 0.18 |
| Shh-N | 0.07 | 0.23 | 0.07 | 0.16 | 0.48 | 0.16 |
| RGM-C | 0.89 | 0.69 | 0.89 | 0.15 | 0.06 | 0.15 |
| Pref-1 | 0.03 | 0.13 | 0.03 | 0.13 | 0.37 | 0.13 |
| CD229 | 0 | 0.02 | 0 | 0.1 | 0.56 | 0.1 |
| Ephrin-B3 | 0.02 | 0.02 | 0.02 | 0.1 | 0.06 | 0.1 |
| Caspase 7 | 0.11 | 0.13 | 0.11 | 0.09 | 0.17 | 0.09 |
| TIM-1 | 0.01 | 0.01 | 0.01 | 0.07 | 0.13 | 0.07 |
| Nectin-1 | 0.28 | 0.1 | 0.28 | 0.06 | 0.02 | 0.06 |
| GFR alpha-2 | 0.14 | 0.37 | 0.14 | 0.06 | 0.21 | 0.06 |
| DCTN1 | 0.01 | 0.04 | 0.01 | 0.05 | 0.04 | 0.05 |
| PCDH17 | 0.33 | 0.03 | 0.33 | 0.05 | 0 | 0.05 |
| BLMH | 0.14 | 0.16 | 0.14 | 0.04 | 0.05 | 0.04 |
| MIP-3b | 0.01 | 0.02 | 0.01 | 0.04 | 0.03 | 0.04 |
| Calsyntenin-1 | 0.01 | 0.1 | 0.01 | 0.03 | 0.19 | 0.03 |
| LDL R | 0 | 0 | 0 | 0.03 | 0.01 | 0.03 |
| IL-13 R2 | 0.01 | 0.02 | 0.01 | 0.03 | 0.04 | 0.03 |
| Thrombospondin-2 | 0.27 | 0.04 | 0.27 | 0.03 | 0 | 0.03 |
| TFF3 | 0.01 | 0.01 | 0.01 | 0.03 | 0.01 | 0.03 |
| Cripto-1 | 0 | 0 | 0 | 0.02 | 0.03 | 0.02 |
| BAMBI | 0.06 | 0.24 | 0.06 | 0.02 | 0.04 | 0.02 |
| Contactin-4 | 0.03 | 0.01 | 0.03 | 0.02 | 0.01 | 0.02 |
| CD155 | 0.02 | 0.05 | 0.02 | 0.02 | 0.05 | 0.02 |
| FGF-20 | 0.53 | 0.58 | 0.53 | 0.02 | 0.04 | 0.02 |
| IL-23 | 0.01 | 0.03 | 0.01 | 0.02 | 0.02 | 0.02 |
| FABP6 | 0 | 0.1 | 0 | 0.02 | 0.24 | 0.02 |
| GITR L | 0.04 | 0.09 | 0.04 | 0.01 | 0.02 | 0.01 |
| IL-22 | 0.02 | 0.15 | 0.02 | 0.01 | 0.11 | 0.01 |
| aFGF | 0.07 | 0.1 | 0.07 | 0.01 | 0.01 | 0.01 |
| GDF-11 | 0.21 | 0.28 | 0.21 | 0.01 | 0.02 | 0.01 |
| Legumain | 0.07 | 0.02 | 0.07 | 0.01 | 0 | 0.01 |
| SLPI | 0.02 | 0.01 | 0.02 | 0.01 | 0 | 0.01 |
| PCK1 | 0.11 | 0.33 | 0.11 | 0 | 0.04 | 0 |
| CXADR | 0 | 0 | 0 | 0 | 0 | 0 |
| DLL1 | 0 | 0 | 0 | 0 | 0 | 0 |
| C1qTNF1 | 0 | 0 | 0 | 0 | 0 | 0 |
| MCP-2 | 0 | 0 | 0 | 0 | 0 | 0 |
| Neuropilin-1 | 0 | 0 | 0 | 0 | 0 | 0 |
| ANG-4 | 0.13 | 0.19 | 0.13 | 0 | 0 | 0 |
| Hepassocin | 0 | 0 | 0 | 0 | 0 | 0 |
| MBL | 0 | 0 | 0 | 0 | 0 | 0 |
| Dkk-3 | 0 | 0.02 | 0 | 0 | 0 | 0 |
| LRRC4 | 0.01 | 0.02 | 0.01 | 0 | 0 | 0 |
| Cf10 | 0 | 0.01 | 0 | 0 | 0 | 0 |
| Siglec-5 | 0 | 0 | 0 | 0 | 0 | 0 |
| CD40L | 0 | 0 | 0 | 0 | 0 | 0 |
| DR6 | 0 | 0 | 0 | 0 | 0 | 0 |
| Angiostatin | 0 | 0 | 0 | 0 | 0 | 0 |

### Example 5

FIG. 6A presents a bubble plot illustrating the association of multiple biomarkers with clinical outcomes, expressed as Hazard Ratio (y-axis) versus −log 10(p-value) (x-axis). Each bubble represents a biomarker, with bubble size proportional to its effect magnitude or statistical weight. The key findings in FIG. 6A are as follows. Tenascin C shows the largest bubble and highest hazard ratio (>4) with strong statistical significance (far right on x-axis), indicating it is the most influential predictor of adverse outcome. Other notable markers include CD206, Fetuin B, MCP-1, and CD163, all positioned above hazard ratio 2 and with significant p-values, suggesting strong associations with risk. Biomarkers such as nidogen-1, IL-6, etc. exhibit moderate hazard ratios—(about 1.5-2) and remain statistically significant. The majority of features cluster near hazard ratio 1 with lower −log 10(p-values), indicating minimal or no association with outcome.

FIG. 6B presents a bubble plot illustrating the association of multiple biomarkers with clinical outcomes, expressed as Hazard Ratio (y-axis) versus −log 10(p-value) (x-axis). Each bubble represents a biomarker, with bubble size proportional to its effect magnitude or statistical weight. The key findings in FIG. 6B are as follows. Tenascin C shows the largest bubble and highest hazard ratio (>4) with strong statistical significance (far right on x-axis), indicating it is the most influential predictor of adverse outcome. Other notable markers include C1qR1, CD206, NCAM-1, and CD163, all positioned above hazard ratio 2 and with significant p-values, suggesting strong associations with risk. Biomarkers such as nidogen-1, IL-6 etc. exhibit moderate hazard ratios (about 1.5-2) and remain statistically significant. The majority of features cluster near hazard ratio 1 with lower −log 10(p-values), indicating minimal or no association with outcome.

FIG. 7 presents a bubble plot illustrating the association of multiple biomarkers with clinical outcomes, expressed as Hazard Ratio (y-axis) versus −log 10(p-value) (x-axis). Each bubble represents a biomarker, with bubble size proportional to its effect magnitude or statistical weight. The key findings in FIG. 7 are as follows. Tenascin C shows the largest bubble and highest hazard ratio (>4) with strong statistical significance (far right on x-axis), indicating it is the most influential predictor of adverse outcome. Other notable markers include CD206, NCAM, C1qR1, and CD163, all positioned above hazard ratio 2 and with significant p-values, suggesting strong associations with risk. Biomarkers such as IL-6, MCP-1, and DR6 exhibit moderate hazard ratios (about 1.5-2) and remain statistically significant. The majority of features cluster near hazard ratio 1 with lower −log 10(p-values), indicating minimal or no association with outcome.

### Example 6

Kidney survival is the ultimate treatment goal in lupus nephritis (LN), but long-term predictors remain understudied due to the need for extensive follow up. Proteinuria at one year is used to establish treatment response, but it is inadequate: repeat biopsy studies showed persistent intrarenal activity in about 50% of proteinuric responders with up to 30% progressing to renal insufficiency. Our objective was to identify urinary biomarkers that predict kidney function loss.

We followed 170 LN patients from the Accelerating Medicines Partnership (AMP) cohort up to 7.8 years (median 4.9). Treatment was discretionary. Estimated Glomerular Filtration Rate (eGFR) loss was defined as a sustained ≥40% decline in eGFR from baseline or development of end-stage kidney disease. We quantified 1200 biomarkers (RayBiotech) in urine samples collected at time of kidney biopsy and month 3, 6, and 12 in LN. Time-to-event models were used to evaluate associations between urinary protein levels and risk of future eGFR loss. Kidney single-cell RNA-seq and spatial transcriptomics (Xenium) were used to identify the renal cellular source of biomarkers. A Random Survival Forest model was trained to predict eGFR loss at any time after baseline and validated on a 20% hold-out test set.

During follow-up, 53/170 (31%) patients developed eGFR loss. At month 3, tenascin C emerged as the strongest predictor of eGFR loss (HR 4.6) and remained elevated through month 12. Several inflammatory and fibrosis-associated markers (i.e., CD163, CD206, FABP4, IL6, IGFBP-6) were persistently associated with increased risk. Single-cell and spatial transcriptomics localized tenascin C expression to interstitial myofibroblasts in LN kidneys, suggesting a pathologic role in progressive fibrosis. To maximize clinical applicability, we developed a urinary protein-based classifier to predict future eGFR loss at any time point after baseline. The model (11-proteins, including tenascin C) achieved excellent predictive performance (AUC=0.91 at 48 months) stratifying patients into high- and low-risk groups. For example, at month 12, the risk score was significantly associated with eGFR preservation (p<0.001), and revealed heterogeneity within proteinuric response groups: some responders (UPCR<0.5) had high risk scores, while some non-responders had low scores. The classifier accurately predicted eGFR loss in both groups underscoring the limitations of proteinuria and the added utility of biomarker-based risk prediction.

Urinary tenascin C, a marker of myofibroblast activation, emerged as a robust predictor of kidney function loss in LN. Elevated CD163 and CD206 levels in high-risk patients point to ongoing M2 macrophage-driven profibrotic signaling and incomplete resolution of intrarenal inflammation, even among proteinuric responders. A proteomics-based risk score outperformed proteinuria, accurately stratifying high-risk individuals regardless of response. These findings offer mechanistic insight and support early biomarker-guided strategies to personalize treatment, refine clinical trials, and prevent irreversible kidney damage.

### Example 7

Risk Stratification and Survival Analysis. A cohort of 274 subjects diagnosed with a chronic disease was evaluated using a proprietary risk scoring algorithm. Subjects were classified into two groups: high-risk (n=145) and low-risk (n=129). Kaplan-Meier survival analysis was performed over a 100-month observation period. Survival probability was plotted against time for each group, and confidence intervals were calculated using Greenwood's formula.

The high-risk group exhibited a progressive decline in survival probability, reaching approximately 0.25 at 100 months, whereas the low-risk group maintained survival above 0.75 throughout the observation period. A log-rank test comparing the two curves yielded a p-value <0.0001, indicating a statistically significant difference in survival outcomes between groups.

The graphical representation of the survival curves, including shaded confidence intervals and tabulated numbers at risk at intervals of 0, 25, 50, 75, and 100 months, was generated by the system described herein. This example demonstrates the utility of the invention for prognostic evaluation and visualization of survival outcomes based on risk classification.

### Example 8

We began with a comprehensive evaluation of approximately 1,200 protein biomarkers measured on the RayBiotech proteomic array. The goal was to identify markers associated with risk of eGFR decline over longitudinal follow-up.

Cox Volcano Analysis Across Three Time Points. At three discrete follow-up intervals from baseline, 3 months, 6 months, and 12 months, we performed univariate Cox proportional hazards modeling for every protein in the array. Each model estimated a hazard ratio (HR) quantifying the association between protein expression and subsequent kidney function decline, along with a p-value for statistical significance. The results for each time point were visualized using volcano plots, where the x-axis represents the log(HR), reflecting effect size. The y-axis represents the −log 10(FDR-adjusted p-value), highlighting statistical strength after correction for multiple testing. From these analyses, we prioritized biomarkers that demonstrated: Highly significant FDR-adjusted p-values, indicating robust statistical association; and Hazard ratios >1, meaning higher protein levels were associated with increased risk of eGFR loss. This yielded a list of top-performing markers (“cox volcano features”) across the three follow-up windows.

Integration With Time-to-Event Features From Predictive Models. To broaden the feature set beyond univariate associations, we also collected top time-to-event (TTE) features identified across top models and all other survival modeling runs. Taking the union of the cox volcano features and the aggregated TTE features produced a combined list of 47 candidate biomarkers shown in Table 5 for deeper evaluation.

TABLE 5

| | Average | TTE | Volcano Plot |
|---|---|---|---|
| Protein | importance | (Random | (Cox |
| (feature) | score | Survival Forest) | Analysis) |
| MIP_1d | 5.133485758 | X | |
| Tenascin C | 4.692011011 | X | X |
| IL_1ra | 4.026079477 | X | |
| MMR | 3.635201898 | X | X |
| LIMPII | 3.206333782 | | X |
| IGFBP_6 | 3.177972452 | X | X |
| Siglec_5 | 3.064797603 | X | X |
| ANGPTL4 | 3.019464706 | X | |
| ESAM | 2.99734682 | X | |
| NPDC_1 | 2.980596845 | X | X |
| EphA1 | 2.927476054 | X | |
| JAM_A | 2.818745377 | X | |
| MCP_1 | 2.788508381 | X | X |
| TIM_1 | 2.760884071 | X | X |
| TIMP_1 | 2.689623817 | X | |
| MCP_2 | 2.659933017 | X | |
| ANG_2 | 2.600340166 | X | |
| DLL1 | 2.561678884 | | X |
| IL_6 | 2.510006097 | | X |
| Notch_3 | 2.509232998 | X | X |
| FABP4 | 2.410678256 | X | |
| Cf10 | 2.348519515 | | X |
| CD163 | 2.239576444 | X | X |
| MCSF R | 2.205815385 | X | |
| Dkk_3 | 2.200068958 | X | X |
| DR6 | 2.111562554 | | X |
| ADAM9 | 2.104527369 | | X |
| SLPI | 2.010984535 | X | |
| PIGF_2 | 1.974749304 | | X |
| Angiostatin | 1.959600346 | | X |
| HCC_4 | 1.865953663 | X | |
| Cathepsin B | 1.832828387 | | X |
| MIP_3b | 1.816791237 | X | |
| FABP2 | 1.775639492 | | X |
| CXCL16 | 1.729463626 | | X |
| LAP(TGFb1) | 1.715927176 | | X |
| C1qTNF1 | 1.684692256 | | X |
| NCAM_1 | 1.668784352 | | X |
| Cyclophilin A | 1.573778743 | | X |
| TSK | 1.55508238 | | X |
| Hepsin | 1.493671149 | | X |
| Nestin | 1.458659027 | | X |
| PCSK9 | 1.310110157 | | X |
| Fetuin B | 1.278748832 | | X |
| Neudesin | 1.009833566 | | X |
| Siglec_11 | 0.877916353 | | X |

Random Survival Forest Analysis (Surv_Ranger). To assess multivariate importance and capture nonlinear relationships, we applied a random survival forest approach using the Surv_Ranger implementation. Random survival forests extend the standard random forest algorithm to handle censored time-to-event outcomes, enabling the model to detect nonlinear effects, capture high-order interactions, and provide ensemble-based importance measures not available in traditional Cox models. Because the feature space was relatively large compared to sample size, we used an iterative cross-validation strategy to limit overfitting and stabilize importance rankings.

Ranking Features by Average Permutation Importance. We ran the Surv_Ranger model 25 times, each with different cross-validation splits. For each run, the permutation importance metric was extracted for all 47 features.

We then averaged the permutation importance scores across all 25 runs, generating a stable measure of each biomarker's contribution to predictive performance. Flagged features that overlapped with the original TTE list or the volcano plot (Cox Analysis) list-highlighting biomarkers that were prioritized both by classical survival analysis and machine-learning-based methods. We then tested permutations of highly ranked features in various combinations as detailed in the series of Kaplan Meier figures optimizing for models that demonstrate high performance in terms of C-index and ability to significantly predict eGFR loss.

### Example 9

A cohort of 274 subjects diagnosed with lupus nephritis (LN) was evaluated using a machine learning approach. To assess multivariate importance and capture nonlinear relationships, we applied a random survival forest approach using the Surv_Ranger implementation in R. Random survival forests extend the standard random forest algorithm to handle censored time-to-event outcomes, enabling the model to detect nonlinear effects, capture high-order interactions, and provide ensemble-based importance measures not available in traditional Cox models. Because the feature space was relatively large compared to sample size, we used an iterative cross-validation strategy to limit overfitting and stabilize importance rankings.

A random survival forest analysis Incorporating the biomarkers tenascin C, CD163, DR6, DLL1, CD206, IGFBP-6, and IL-6 was performed to stratify patients into low and high risk for future kidney functional loss (eGFR loss >40% greater than baseline). Based on the resulting composite score, subjects were stratified into high-risk (n=145) and low-risk (n=129) groups. Model performance was assessed using the concordance index (C-index), with a median C-index of 0.79 across multiple iterative cross-validations, indicating strong discriminative ability in predicting renal survival.

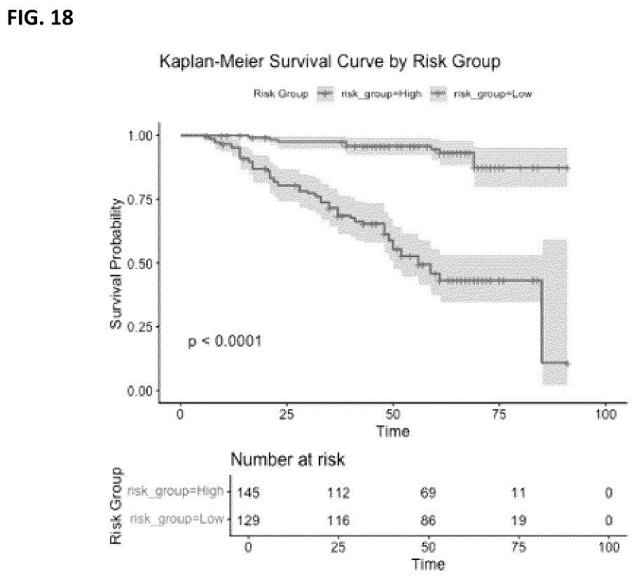

Kaplan-Meier survival analysis was conducted over a 100-month observation period to compare outcomes between the two groups. Survival probability was plotted as a function of time, with 95% confidence intervals estimated using Greenwood's formula. The high-risk group demonstrated a marked, progressive decline in survival probability, reaching approximately 0.25 by 100 months. In contrast, the low-risk group maintained survival probabilities above 0.75 across the entire follow-up period. A log-rank test comparing the two curves yielded a p-value <0.0001, confirming a highly significant difference in renal survival between groups. The system generated the Kaplan-Meier plot shown in FIG. 18 with shaded confidence intervals and included a table of numbers at risk at 0, 25, 50, 75, and 100 months. This example highlights the utility of the invention for prognostic assessment and visualization of long-term survival outcomes based on a biomarker-driven risk classification model.

### Example 10

A cohort of 274 subjects diagnosed with lupus nephritis (LN) was evaluated using a machine-learning-based prognostic modeling approach. To assess multivariate feature importance and capture nonlinear relationships, we applied a random survival forest method using the Surv_Ranger implementation in R. Random survival forests generalize the standard random forest algorithm to censored time-to-event data, enabling the detection of nonlinear effects, higher-order interactions, and ensemble-derived importance measures that exceed the capabilities of traditional Cox models. Because the feature space was relatively large compared with the sample size, we employed an iterative cross-validation strategy to reduce overfitting risk and stabilize importance rankings.

A random survival forest analysis incorporating the biomarkers tenascin C, MIP-1d (CCL15), IL-1Ra, CD206, and IGFBP-6 was used to generate a composite risk score predicting future kidney functional decline (defined as eGFR loss >40% from baseline). Based on this score, subjects were stratified into high-risk (n=141) and low-risk (n=133) groups. Model performance was evaluated using the concordance index (C-index), with a median C-index of 0.77 across multiple iterative cross-validations, demonstrating strong discriminative ability for predicting renal outcomes.

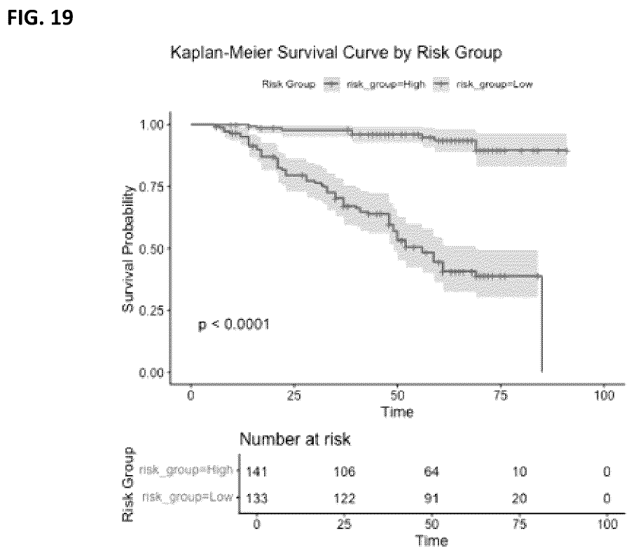

Kaplan-Meier survival analysis was performed over a 100-month observation window to compare renal survival between the two groups. Survival probability was plotted as a function of time, and 95% confidence intervals were calculated using Greenwood's formula. The high-risk group exhibited a pronounced, progressive decline in survival probability, decreasing to approximately 0.40 at 80 months. In contrast, the low-risk group maintained survival probabilities above 0.75 for the duration of follow-up. A log-rank test comparing the survival curves yielded a p-value <0.0001, confirming a highly significant separation in renal survival between groups. The system automatically generated the Kaplan-Meier curves show in FIG. 19 with shaded confidence intervals and produced a table of numbers at risk at 0, 25, 50, 75, and 100 months. This example demonstrates the utility of the invention for prognostic evaluation, risk stratification, and visualization of long-term survival outcomes using a biomarker-driven machine learning model.

### Example 11

A cohort of 274 subjects diagnosed with lupus nephritis (LN) was evaluated using a machine-learning-based prognostic modeling approach. To assess multivariate feature importance and capture nonlinear relationships, we applied a random survival forest method using the Surv_Ranger implementation in R. Random survival forests generalize the standard random forest algorithm to censored time-to-event data, enabling the detection of nonlinear effects, higher-order interactions, and ensemble-derived importance measures that exceed the capabilities of traditional Cox models. Because the feature space was relatively large compared with the sample size, we employed an iterative cross-validation strategy to reduce overfitting risk and stabilize importance rankings.

A random survival forest analysis incorporating the biomarkers tenascin C, LIMP-2, Siglec-5, ANGPTL4, and ESAM. Based on the resulting composite score, subjects were stratified into high-risk (n=136) and low-risk (n=138) groups. Model performance was evaluated using the concordance index (C-index), with a median C-index of 0.77 across multiple iterative cross-validations, demonstrating strong discriminative ability for predicting renal outcomes.

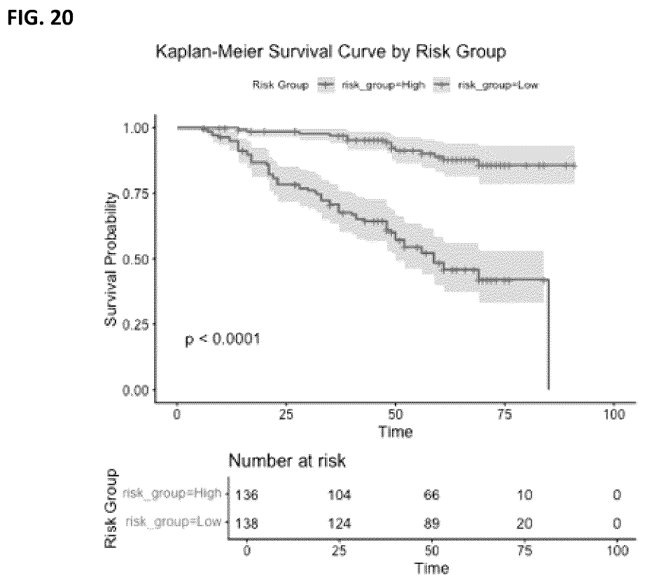

Kaplan-Meier survival analysis was performed over a 100-month observation period to assess differences in renal survival between the two groups. Survival probability was plotted as a function of time, and 95% confidence intervals were estimated using Greenwood's formula. The high-risk group demonstrated a progressive decline in survival probability, falling to approximately 0.40 at 80 months. In contrast, the low-risk group maintained renal survival above 0.75 throughout the entire follow-up interval. A log-rank test comparing the two survival curves yielded a p-value <0.0001, indicating a highly significant difference in renal outcomes between risk groups. The Kaplan-Meier curves shown in FIG. 20, including shaded confidence intervals and tabulated numbers at risk at 0, 25, 50, 75, and 100 months, were generated by the system described herein. This example illustrates the utility of the invention for prognostic evaluation, risk stratification, and visualization of long-term survival outcomes using a biomarker-driven risk classification model.

### Example 12

A cohort of 274 subjects diagnosed with lupus nephritis (LN) was evaluated using a proprietary multivariate risk-scoring algorithm incorporating the biomarkers tenascin C, MCP-1, TIM-1, TIMP-1, DLL-1, NOTCH-3, and CD163. Based on the resulting composite score, subjects were stratified into high-risk (n=135) and low-risk (n=139) groups.

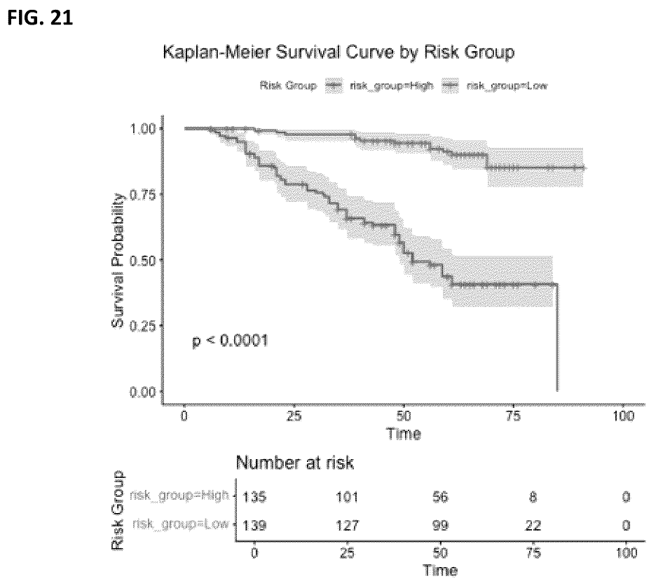

Model performance was quantified using the concordance index (C-index), with a median C-index of 0.79, indicating strong discriminative ability for predicting long-term renal survival. Kaplan-Meier survival analysis was conducted over a 100-month observation period to compare renal outcomes between the two groups. Survival probability was plotted as a function of time, with 95% confidence intervals calculated using Greenwood's formula. The high-risk group demonstrated a steady decline in renal survival, reaching approximately 0.40 by 80 months. In contrast, the low-risk group maintained survival probabilities above 0.75 throughout the entire follow-up interval. A log-rank test comparing the two curves yielded a p-value <0.0001, confirming a highly significant separation in survival outcomes between the two strata. The Kaplan-Meier curves shown in FIG. 21, complete with shaded confidence intervals and tabulated numbers at risk at 0, 25, 50, 75, and 100 months, were generated by the system described herein. This example demonstrates the utility of the invention for prognostic evaluation, risk stratification, and visualization of long-term renal survival using a biomarker-driven predictive model.

### Example 13

A cohort of 274 subjects diagnosed with lupus nephritis (LN) was evaluated using a proprietary multivariate risk-scoring algorithm. Based on the resulting composite score, subjects were stratified into high-risk (n=135) and low-risk (n=139) groups.

Model performance was assessed using the concordance index (C-index), with a median C-index of 0.76, indicating strong discriminative ability for predicting long-term renal survival.

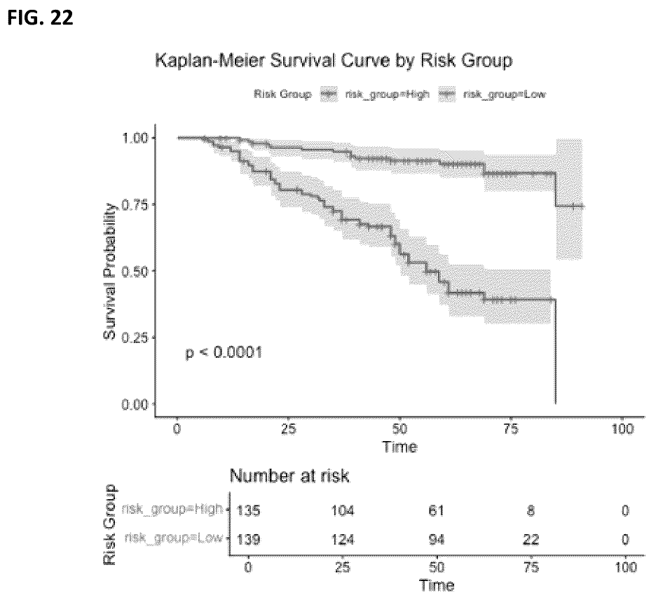

Kaplan-Meier survival analysis was performed over a 100-month observation period to compare renal outcomes between groups. Survival probability was plotted as a function of time, with 95% confidence intervals estimated using Greenwood's formula. The high-risk group exhibited a progressive decline in survival probability, reaching approximately 0.40 by 80 months, whereas the low-risk group maintained survival probabilities at or above 0.75 throughout the observation period. A log-rank test comparing the two curves yielded a p-value <0.0001, indicating a statistically significant difference in renal survival between risk strata. The system generated the Kaplan-Meier curves shown in FIG. 22 with shaded confidence intervals and included a table of numbers at risk at 0, 25, 50, 75, and 100 months. This example demonstrates the utility of the invention for prognostic evaluation, risk stratification, and visualization of long-term survival outcomes based on a biomarker-driven risk classification model.

It is understood that the examples described herein are for illustrative purposes only and that various modifications or changes in light thereof will be suggested to persons skilled in the art and are to be included within the spirit and scope of this application and claims. The section headings used herein are for organizational purposes only and are not to be construed as limiting the subject matter described. All documents, or portions of documents, cited in the application are expressly incorporated by reference herein in their entirety and for all purposes.

1. A method of detecting a protein in a human patient having lupus nephritis, the method comprising detecting:
(a) an elevated level of a protein, relative to a control, in a biological sample obtained from the patient, wherein the protein comprises CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, or a combination of two or more thereof, and/or
(b) a reduced level of a protein, relative to a control, in the biological sample obtained from the patient, wherein the protein comprises C5a, FOLR2, PRX1, P-cadherin, NCAM-1, IFN-gamma R1, HNMT, galectin-4, fetuin B, CRTAM, MFRP, SULTIB1, or a combination of two or more thereof.

2. A method of treating lupus nephritis in a human patient in need thereof, the method comprising:
(i) detecting:
(a) an elevated level of a protein, relative to a control, in a biological sample obtained from the patient, wherein the protein comprises CD163, PRTN3, catalase, CEACAM-1, IP-10, IL-16, cathepsin S, cyclophilin A, FKBP51, S100A13, galectin-1, MMR, S100A8, CES1, nidogen-1, IL-6, CrkL, midkine, MIP-1b, tenascin C, C1q R1, PAM, PCSK9, ADAM8, CXCL16, nestin, galectin-8, or a combination of two or more thereof, and/or
(b) a reduced level of a protein, relative to a control, in the biological sample obtained from the patient, wherein the protein comprises C5a, FOLR2, PRX1, P-cadherin, NCAM-1, IFN-gamma R1, HNMT, galectin-4, fetuin B, CRTAM, MFRP, SULT1B1, or a combination of two or more thereof,
(ii) administering to the human patient an effective amount of a lupus nephritis therapeutic agent.

3-128. (canceled)

129. The method of claim 1, wherein the protein comprises tenascin C.

130. The method of claim 1, wherein the protein comprises tenascin C and a protein selected from the group consisting of CD163, PRTN3, and MMR.

131. The method of claim 1, wherein the protein comprises tenascin C, CD163, PRTN3, and MMR.

132. The method of claim 1, wherein the protein comprises tenascin C, CD163, PRTN3, MMR, or a combination of two or more thereof.

133. The method of claim 1, further comprising detecting an elevated level of a protein, relative to a control, in a biological sample obtained from the patient, wherein the protein comprises azurocidin, thyroglobulin, DBH, visfatin, DR6, DLL1, IGFBP-6, MIP-1d, IR-1Ra, Siglec-5, LIMP-2, ANGPTL4, ESAM, MCP-1, TIM-1, TIMP-1, NOTCH-3, or a combination of two or more thereof.

134. A method of detecting a protein in a human patient having lupus nephritis, the method comprising detecting an elevated level of a protein, relative to a control, in a biological sample obtained from the patient, wherein the protein comprises CD163, IL-16, azurocidin, tenascin C, IL-16, catalase, thyroglobulin, S100A8, PRTN3, DBH, visfatin, DR6, DLL1, MMR, IGFBP-6, IL-6, MIP-1d, IL-1Ra, LIMP-2, Siglec-5, ANGPTL4, ESAM, MCP-1, TIM-1, TIMP-1, NOTCH-3, or a combination of two or more thereof.

135. The method of claim 134, wherein the protein comprises IL-16, CD163, azurocidin, thyroglobulin, catalase, tenascin C, S100A8, DBH, visfatin, PRTN3, or a combination of two or more thereof.

136. The method of claim 1, comprising detecting the level of a protein via enzyme-linked immunosorbent assay.

137. The method of claim 1, further comprising detecting:
(a) an elevated level of complement component 3 (C3), relative to a control, in the biological sample obtained from the subject;
(b) an elevated level of complement component 4 (C4), relative to a control, in the biological sample obtained from the subject;
(c) an elevated level of anti-dsDNA, relative to a control, in the biological sample obtained from the subject;
(d) an elevated level of proteinuria, relative to a control, in the biological sample obtained from the subject; or
(e) a combination of two or more of (a), (b), (c), and (d).

138. The method of claim 1, wherein the human patient has an NIH Activity Index greater than 2.

139. The method of claim 1, wherein the lupus nephritis is histologically active lupus nephritis.

140. The method of claim 1, wherein the lupus nephritis is proliferative lupus nephritis, membranous lupus nephritis, or mixed lupus nephritis.

141. The method of claim 1, wherein the lupus nephritis is Class I lupus nephritis, Class II lupus nephritis, Class II lupus nephritis, Class II lupus nephritis, Class II lupus nephritis, or Class II lupus nephritis.

142. The method of claim 1, wherein the human patient has systemic lupus erythematosus.

143. The method of claim 1, wherein the biological sample is urine.

144. The method of claim 1, wherein the control is a patient with histologically inactive lupus nephritis, a patient with nonproliferative lupus nephritis, or a healthy patient.

145. The method of claim 2, wherein the lupus nephritis therapeutic agent is an immunosuppressive agent, a B-cell inhibitor, a B-cell targeting treatment, an interferon antagonist, a glucocorticoid, or a combination of two or more thereof.

146. The method of claim 145, wherein:
(i) the immunosuppressive agent is tacrolimus, methotrexate, leflunomide, hydroxychloroquine, azathioprine, cyclosporine, cyclophosphamide, mycophenolate, or voclosporin;
(ii) the B-cell inhibitor is belimumab, rituximab, ocrelizumab, veltuzumab, obinutuzumab, ibritumomab tiuxetan, ofatumumab, or epratuzumab;
(iii) the interferon antagonist is anifrolumab, IFN-α kinoid, rontalizumab, sifalimumab, or bortezomib;
(iv) the glucocorticoid is prednisone, methylprednisolone, dexamethasone, or betamethasone; and
(v) the B-cell targeting treatment is a cellular therapy including but not limited to anti-CD19, anti-CD20, anti-BCMA, CAR-T cells, or T cell engagers.
